(19) United States
(12) Patent Application Publication
(10) Pub. No.: US 2003/0170627 A1
(43) Pub. Date: Sep. 11, 2003

(54) cDNAs co-expressed with placental steroid synthesis genes

(76) Inventors: Michael G. Walker, Sunnyvale, CA (US); Sophia Li-Ming Wong, San Jose, CA (US)

Correspondence Address: INCYTE CORPORATION (formerly known as Incyte, Genomics, Inc.), 3160 PORTER DRIVE, PALO ALTO, CA, 94304, US

(21) Appl. No.: 09/996,952

(22) Filed: Nov. 27, 2001

Related U.S. Application Data
(60) Provisional application No. 60/253,425, filed on Nov. 27, 2000.

(51) Int. Cl. C12Q 1/68; C07H 21/04; C12P 21/02; C12N 5/06; C12N 9/02; C12N 9/10

(52) U.S. Cl. 435/6; 435/189; 435/193; 435/69.1; 435/320.1; 435/325; 536/23.2

(57) ABSTRACT

The invention provides novel cDNAs that coexpress with known placental steroid synthesis genes. The invention also provides expression vectors, host cells, proteins encoded by the cDNAs and antibodies which specifically bind the proteins. The invention also provides methods for the diagnosis, prognosis, evaluation of therapies and treatment of disorders associated with steroid-responsive tissues and with pregnancy.

This application claims the benefit of provisional application No. 60/253,425, filed Nov. 27, 2000.

### FIELD OF THE INVENTION

The invention relates to cDNAs identified by their coexpression with known placental steroid synthesis genes and to their use in the diagnosis, prognosis, treatment, and evaluation of therapies for disorders associated with steroid-responsive tissues and with pregnancy.

### BACKGROUND OF THE INVENTION

During pregnancy, the placenta is the primary source of the steroid hormones, estrogen and progesterone (Nestler 1990). The cells and tissues of the placenta express a variety of genes that regulate or participate in steroid synthesis. These include aromatase P-450, cholesterol side-chain cleavage enzyme, meltrin-L, placental lactogen hormone, pregnancy-associated plasma protein-A, hydroxysteroid dehydrogenase, pregnancy-specific beta-glycoprotein, and placental alkaline phosphatase. The expression of these genes and the proteins which they encode play an important role in pregnancy, both in the normal development of the fetus and in disorders which affect the fetus and/or the mother, particularly pregnancy-induced hypertension (PIH) and preeclampsia.

PIH is a major source of morbidity and mortality in both mothers and fetuses (Jain (1997) J Perinatol 17:425-427; Innes and Wimsatt (1999) Acta Obstet Gynecol Scand 78:263-284; and Visser and Wallenburg (1999) Baillieres Best Pract Res Clin Obstet Gynaecol 13:131-156), but both the origin and the progression of PIH are poorly understood. Prevention requires that patients at risk be identified, but even though numerous predictive methods have been published, few have proven useful (Visser and Wallenburg, supra).

Preeclampsia, a condition of late pregnancy which is characterized by hypertension, edema, and proteinuria, may lead to the convulsions and coma of eclampsia. Although many interventions have been proposed, none have shown efficacy in large, multi-center controlled trials, and few have shown efficacy in more limited trials (Visser and Wallenburg, supra).

The role of altered synthesis of steroids is significant in PIH. A mutation in the mineralocorticoid receptor (MR), S810L, causes early-onset hypertension that is markedly exacerbated in pregnancy. This mutation results in constitutive MR activity and alters receptor specificity. This alteration causes progesterone and other steroids that are normally MR antagonists to become potent MR agonists. The resulting increase in salt and water retention induces hypertension (Geller et al. (2000) Science 289:119-123). There is some indication that the effect of catechol estrogen on placental steroidogenesis may be related to PIH in that estrogen 2-hydroxylase activity is significantly higher in PIH placenta than in normal placenta (Okubo et al. (1996) Endocr J 43:363-368).

Gestational hypertension is associated with diabetes and insulin resistance. Controlled studies by at least 11 different research groups have shown significant positive association between PIH and various measures of insulin resistance. Hyperinsulinemia, glucose intolerance, and insulin insensitivity all predict the subsequent development of PIH (Valensise et al. (1996) Diabetologia 39:952-960; Hadden (1999) Diabetes Care 22(s2):B 104-108; and Innes and Wimsatt, supra). Pregnancy-induced diabetes is associated with alterations in placental steroid synthesis. Insulin and the insulin-like growth factors regulate placental steroidogenesis by modulating the activity of the placental steroid-synthesis enzymes aromatase P450, 3-beta hydroxysteroid dehydrogenase, and cholesterol side-chain cleavage enzyme (Nestler (1987) Endocrinology 121:1845-1852; Nestler (1989) Endocrinology 125:2127-2133; Nestler et al. (1991) Endocrinology 129:2951-2956; and Nestler (1993) J Steroid Biochem Mol Biol 44:449-457). Pregnant diabetic women experience significant abnormalities in estrogen and progesterone levels compared to non-diabetic pregnant women (Nestler (1987, supra); Diamant, (1991) Isr J Med Sci 27:493-497; and Olszewski et al. (1995) Ginekol Pol 66:145-150).

In addition to hypertension and diabetes, placental steroid synthesis genes play a role in the expression and/or activity of other pregnancy-associated and placental-associated genes. In women at risk for fetal growth retardation (FGR), higher levels of estradiol, placental lactogen, and pregnancy specific beta-1 glycoprotein (PS beta-1Gp) are associated with a decreased prevalence of FGR (Gardner et al. (1997) Acta Obstet Gynecol Scand Suppl 165:56-58). Determination of placental alkaline phosphatase (PLAP) is used in detecting damage to fetal alveolar cells caused by maternal smoking (Xie et al. (1996) Chung Hua Cheng Hsing Shao Shang Wai Ko Tsa Chih 12:427-430). Other potential uses for PS beta-1Gp and PLAP are as biochemical markers that predict successful implantation of human blastocycts after in vitro fertilization (Jurisicova et al. (1999) Hum Reprod 14:1852-1858). Another marker is pregnancy-associated plasma protein A which, when reduced during pregnancy, is diagnostic of Down syndrome pregnancy (Qin et al. (1997) Clin Chem 43:2323-2332; De Biasio et al. (1999) Prenat Diagn 19:360-363; and Spencer (1999) Prenat Diagn 19:1065-1066).

The discovery of cDNAs which coexpress with known placental steroid synthesis genes satisfies a need in the art by providing new compositions which are useful in the diagnosis, prognosis, treatment, and evaluation of therapies for disorders associated with steroid-responsive tissues and with pregnancy.

### SUMMARY OF THE INVENTION

The invention provides a combination of isolated cDNAs having the nucleic acid sequences of SEQ ID NOs:1-9 or their complements that are coexpressed with one or more known placental steroid synthesis genes in a plurality of biological samples. In one aspect, the cDNAs comprising the nucleic acid sequences of SEQ ID NOs:1-9 or the complements thereof are used as probes. The invention also provides a cDNA comprising a nucleic acid sequence of SEQ ID NOs:4, 6, and 7 or the complements thereof. The invention further provides an expression vector comprising a cDNA and a host cell containing the expression vector that can be used to produce a protein. The invention still further provides a method for using a cDNA to produce a protein comprising culturing the host cell containing the cDNA under conditions for expression of the protein and recovering the protein from cell culture.

The invention provides a method of using a cDNA of the invention to screen a plurality of molecules to identify at least one ligand which specifically binds the cDNA, the method comprising combining a cDNA selected from the combination with a plurality of molecules under conditions to allow specific binding, and detecting specific binding, thereby identifying a ligand which specifically binds the cDNA. In one aspect, the plurality of molecules is selected from DNA molecules, RNA molecules, peptide nucleic acids, mimetics, and proteins. The invention also provides a method of using a cDNA to purify a ligand that specifically binds the cDNA, the method comprising combining a cDNA selected from the combination with a sample under conditions to allow specific binding, recovering the bound cDNA, and separating the ligand from the bound cDNA, thereby obtaining purified ligand.

The invention provides a method for using a cDNA to detect differential expression in a sample comprising hybridizing at least one cDNA of the invention to nucleic acids in the sample under conditions to form a hybridization complex and comparing complex formation with standards wherein the comparison indicates differential expression. In one aspect, the nucleic acids of the sample are amplified prior to hybridization. In a second aspect, the cDNAs or nucleic acids are attached to a substrate. In one embodiment, differential expression is diagnostic of a disorder associated with steroid-responsive tissues and with pregnancy.

The invention provides a purified protein encoded by SEQ ID NO:4, 6, or 7 that is coexpressed with one or more known placental steroid synthesis genes in a plurality of biological samples. The invention also provides a method for using a protein to screen a plurality of molecules to identify at least one ligand which specifically binds the protein, the method comprising combining the protein with the plurality of molecules under conditions to allow specific binding, and detecting specific binding between the protein and ligand, thereby identifying a ligand which specifically binds the polypeptide. In one aspect, the plurality of molecules is selected from DNA molecules, RNA molecules, PNAs, mimetics, proteins, agonists, antagonists, and antibodies. The invention further provides a method of using a protein to purify a ligand from a sample, the method comprising combining the protein with a sample under conditions to allow specific binding, recovering the bound protein, and separating the ligand from the bound protein, thereby obtaining purified ligand. The invention still further provides a method for using the protein to produce a polyclonal antibody comprising immunizing a animal with protein under conditions to elicit an antibody response, isolating animal antibodies, attaching the protein to a substrate, contacting the substrate with isolated antibodies under conditions to allow specific binding to the protein, dissociating the antibodies from the protein, thereby obtaining purified polyclonal antibodies. The invention yet still further provides a method for preparing and purifying monoclonal antibodies comprising immunizing a animal with a protein under conditions to elicit an antibody response, isolating antibody producing cells from the animal, fusing the antibody producing cells with immortalized cells in culture to form monoclonal antibody producing hybridoma cells, culturing the hybridoma cells, and isolating from culture monoclonal antibodies which specifically bind the protein.

The invention provides purified polyclonal and monoclonal antibodies which bind specifically to a protein. The invention also provides a method for using an antibody to detect expression of a protein in a sample, the method comprising combining the antibody with a sample under conditions which allow the formation of antibody:protein complexes; and detecting complex formation, wherein complex formation indicates expression of the protein in the sample. In one aspect, the protein or antibody are attached to a substrate. In a second aspect, the amount of complex formation when compared to standards is diagnostic of a disorder associated with steroid-responsive tissues and with pregnancy.

The invention provides an antibody that specifically binds to a protein of the invention, and methods for the diagnosis or treatment of a disorder associated with expression of that protein. The invention further provides a composition comprising a cDNA, a protein or an antibody that specifically binds a protein and a labeling moiety, therapeutic agent, or a pharmaceutical carrier.

### BRIEF DESCRIPTION OF THE SEQUENCE LISTING, FIGURES AND TABLE

The Sequence Listing provides exemplary cDNAs comprising the nucleic acid sequences of SEQ ID NOs:1-9. Each sequence has a sequence identification number (SEQ ID NO) and the Incyte number by which it was first identified.

FIGS. 1A-1D show the translation of SEQ ID NO:4 as produced using MACDNASIS PRO software (Hitachi Software Engineering, South San Francisco Calif.).

FIGS. 2A and 2B show the translation of SEQ ID NO:7 as produced using MACDNASIS PRO software (Hitachi Software Engineering).

Table 1 shows the homologs for SEQ ID NOs:1-9. The first column shows sequence identification number (SEQ ID); the second column, Incyte identification number (Incyte No); the third column, GenBank homolog, description and length; the fourth column, the number of embryonic libraries in which the sequence was expressed (Embryonic Libs); and the fifth column, specific expression of the sequence in embryonic tissue (as a pecentage of all the times it was expressed in all tissues).

### DESCRIPTION OF THE INVENTION

It must be noted that as used herein and in the appended claims, the singular forms “a”, “an”, and “the” include the plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

It is to be understood that this invention is not limited to the particular devices, machines, materials and methods described. Although particular embodiments are described, equivalent embodiments may be used to practice the invention. The described embodiments are provided to illustrate the invention and are not intended to limit the scope of the invention which is limited only by the appended claims.

Definitions

“Array” refers to an ordered arrangement of at least two cDNAs, proteins, or antibodies on a substrate. At least one of the cDNAs, proteins, or antibodies represents a control or standard, and the other, a cDNA, protein, or antibody of diagnostic or therapeutic interest. The arrangement of two to about 40,000 cDNAs, proteins, or antibodies on the substrate assures that the size and signal intensity of each labeled complex, formed between each cDNA and at least one nucleic acid, or antibody:protein complex, formed between each antibody and at least one protein to which the antibody specifically binds, is individually distinguishable.

“cDNA” refers to an isolated polynucleotide, nucleic acid molecule, or any fragment or complement thereof. It may have originated recombinantly or synthetically, may be double-stranded or single-stranded, represents coding and noncoding 3′ or 5′ sequence, and generally lacks introns. It may be combined with carbohydrate, lipids, protein or other materials to perform a particular activity such as diagnosis or form a useful composition for therapy.

The “complement” of a cDNA refers to a nucleic acid molecule which is completely complementary to the cDNA over its full length and which will hybridize to the cDNA or an mRNA under conditions of high stringency.

“Differential expression” refers to an increased or up-regulated or a decreased or down-regulated expression as detected by presence, absence or at least two-fold change in the amount or abundance of a transcribed messenger RNA or translated protein in a sample.

“Disorder associated with pregnancy” refers to any condition, disease or disorder that occurs in an individual at risk as the result of pregnancy including, but not limited to, PIH, preeclampsia, hyperinsulinemia, glucose intolerance, insulin insensitivity, fetal growth retardation, Down syndrome pregnancy, and estrogen-sensitive adenocarcinomas of the breast, ovary and uterus.

“Labeling moiety” refers to any reporter molecule, visible or radioactive label, than can be attached to or incorporated into a cDNA, protein or antibody. Visible labels include but are not limited to anthocyanins, green fluorescent protein (GFP), β glucuronidase, luciferase, Cy3 and Cy5, and the like. Radioactive markers include radioactive forms of hydrogen, iodine, phosphorous, sulfur, and the like.

“Protein” refers to a polypeptide or any portion thereof. A “portion” of a protein refers to that length of amino acid sequence which would retain at least one biological activity, a domain identified by PFAM or PRINTS analysis or an antigenic epitope of the protein identified using Kyte-Doolittle algorithms of the PROTEAN program (DNASTAR).

“Sample” is used in its broadest sense as containing nucleic acids, proteins, antibodies, and the like. A sample may comprise a bodily fluid; the soluble fraction of a cell preparation, or an aliquot of media in which cells were grown; a chromosome, an organelle, or membrane isolated or extracted from a cell; genomic DNA, RNA, or cDNA in solution or bound to a substrate; a biopsy, a cell; a tissue; a tissue print; a fingerprint, buccal cells, skin, or hair; and the like.

“Isolated or purified” refers to a cDNA, protein, or antibody that is removed from its natural environment and that is separated from other components with which it is naturally present.

“Placental steroid synthesis gene” refers to a polynucleotide which has been previously identified as useful in the diagnosis, prognosis, or treatment of disorders associated with steroid-responsive tissues and with pregnancy. The known gene is differentially expressed in tissues from patients at risk for a disorder of the invention when compared with normal expression in any tissue. The known placental steroid synthesis genes of this invention are aromatase P-450, cholesterol side-chain cleavage enzyme, meltrin-L, placental lactogen hormone, pregnancy-associated plasma protein-A, hydroxysteroid dehydrogenase, pregnancy-specific beta-glycoprotein, and placental alkaline phosphatase.

“Specific binding” refers to a special and precise interaction between two molecules which is dependent upon their structure, particularly their molecular side groups. For example, the intercalation of a regulatory protein into the major groove of a DNA molecule or the binding between an epitope of a protein and an agonist, antagonist, or antibody.

“Substrate” refers to any rigid or semi-rigid support to which cDNAs or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

A “transcript image” is a profile of gene transcription activity in a particular tissue at a particular time.

“Variant” refers to molecules that are recognized variations of a cDNA or a protein encoded by the cDNA. Splice variants may be determined by BLAST score, wherein the score is at least 100, and most preferably at least 400. Allelic variants have a high percent identity to the cDNAs and may differ by about three bases per hundred bases. “Single nucleotide polymorphism” (SNP) refers to a change in a single base as a result of a substitution, insertion or deletion. The change may be conservative (purine for purine) or non-conservative (purine to pyrimidine) and may or may not result in a change in an encoded amino acid or its secondary, tertiary, or quaternary structure.

The Invention

The present invention utilizes a method known as Guilt-by-Association (GBA) for identifying cDNAs that are associated with a specific disease, regulatory pathway, subcellular compartment, cell type, tissue type, or species and predicting the function of their encoded proteins (Walker et al. (1999) ISMB pp.282-286; Walker et al. (1999) Genome Res. 9:1198-1203, incorporated herein by reference). In particular, the method identifies cDNAs useful in diagnosis, prognosis, treatment, and evaluation of therapies for disorders associated with steroid-responsive tissues and with pregnancy.

The method provides for the identification of cDNAs that are expressed in a plurality of libraries.

The cDNAs include genes of known or unknown function which are expressed in a specific disease process, subcellular compartment, cell type, tissue type, or species. The expression patterns of genes with known function are compared with those of cDNAs with unknown function to determine whether a specified coexpression probability threshold is met. Through this comparison, a subset of the cDNAs having a high coexpression probability with the known placental steroid synthesis genes can be identified. The high coexpression probability correlates with a particular coexpression probability threshold which is preferably less than 0.001 and more preferably less than 0.00001.

The cDNAs originate from cDNA libraries derived from a variety of sources including, but not limited to, eukaryotes such as human, mouse, rat, dog, monkey, plant, and yeast; prokaryotes such as bacteria; and viruses. These cDNAs can also be selected from a variety of sequence types including, but not limited to, expressed sequence tags (ESTs), assembled polynucleotides, genomic exons, promoters, introns, enhancers, 5′ untranslated regions, and 3′ untranslated regions. To have statistically significant analytical results, the cDNAs need to be expressed in at least five cDNA libraries.

The 1176 cDNA libraries used in the coexpression analysis of the present invention were obtained from adrenal gland, biliary tract, bladder, blood cells, blood vessels, bone marrow, brain, bronchus, cartilage, chromaffin system, colon, connective tissue, cultured cells, embryonic stem cells, endocrine glands, epithelium, esophagus, fetus, ganglia, heart, hypothalamus, immune system, intestine, islets of Langerhans, kidney, larynx, liver, lung, lymph, muscles, neurons, ovary, pancreas, penis, peripheral nervous system, phagocytes, pituitary, placenta, pleurus, prostate, salivary glands, seminal vesicles, skeleton, spleen, stomach, testis, thymus, tongue, ureter, uterus, and the like. The number of cDNA libraries selected can range from as few as 5 to greater than 10,000. Preferably, the number of the cDNA libraries is greater than 1000.

In a preferred embodiment, the cDNAs are assembled from sequence fragments derived from a single transcript. Assembly of the polynucleotide can be performed using sequences of various types including, but not limited to, ESTs, extension of the EST, shotgun sequences from a cloned insert, or full length cDNAs. In a most preferred embodiment, the cDNAs are derived from human sequences that have been assembled using the algorithm disclosed in U.S. Pat. No. 9,276,534, filed Mar. 25, 1999, incorporated herein by reference.

Experimentally, differential expression of the polynucleotides can be evaluated by methods including, but not limited to, differential display by spatial immobilization or by gel electrophoresis, genome mismatch scanning, representational difference analysis, and transcript imaging. Additionally, differential expression can be assessed by microarray technology. These methods may be used alone or in combination.

Known placental steroid synthesis genes expressed in disorders associated with steroid-responsive tissues and with pregnancy. can be selected based on the use of the genes as diagnostic or prognostic markers or as therapeutic targets. Preferably, the known genes include aromatase P-450, cholesterol side-chain cleavage enzyme, meltrin-L, placental lactogen hormone, pregnancy-associated plasma protein-A, hydroxysteroid dehydrogenase, pregnancy-specific beta-glycoprotein, and placental alkaline phosphatase.

The procedure for identifying novel cDNAs that exhibit a statistically significant coexpression pattern with known placental steroid synthesis genes is as follows. First, the presence or absence of a gene sequence in a cDNA library is defined: a gene is present in a cDNA library when at least one cDNA fragment corresponding to that gene is detected in a cDNA sample taken from the library, and a gene is absent from a library when no corresponding cDNA fragment is detected in the sample.

Second, the significance of gene coexpression is evaluated using a probability method to measure a due-to-chance probability of the coexpression. The probability method can be the Fisher exact test, the chi-squared test, or the kappa test. These tests and examples of their applications are well known in the art and can be found in standard statistics texts (Agresti (1990) Categorical Data Analysis, John Wiley & Sons, New York N.Y.; Rice (1988) Mathematical Statistics and Data Analysis, Duxbury Press, Pacific Grove Calif.). A Bonferroni correction (Rice, supra, p. 384) can also be applied in combination with one of the probability methods for correcting statistical results of one gene versus multiple other genes. In a preferred embodiment, the due-to-chance probability is measured by a Fisher exact test, and the threshold of the due-to-chance probability is set preferably to less than 0.001, more preferably to less than 0.00001.

To determine whether two genes, A and B, have similar coexpression patterns, occurrence data vectors can be generated as illustrated below. The presence of a gene occurring at least once in a library is indicated by a one, and its absence from the library, by a zero.

### Occurrence Data for Genes A and B

| | Library 1 | Library 2 | Library 3 | . . . | Library N |
|---|---|---|---|---|---|
| Gene A | 1 | 1 | 0 | . . . | 0 |
| Gene B | 1 | 0 | 1 | . . . | 0 |

For a given pair of genes, the occurrence data in can be summarized in a 2×2 contingency table as illustrated below.

Contingency Table for Co-occurrences of Genes A and B

| | Gene A Present | Gene A Absent | Total |
|---|---|---|---|
| Gene B Present | 8 | 2 | 10 |
| Gene B Absent | 2 | 18 | 20 |
| Total | 10 | 20 | 30 |

The contingency table presents co-occurrence data for gene A and gene B in a total of 30 libraries. Both gene A and gene B occur 10 times in the libraries. The table summarizes and presents: 1) the number of times gene A and B are both present in a library; 2) the number of times gene A and B are both absent in a library; 3) the number of times gene A is present, and gene B is absent; and 4) the number of times gene B is present, and gene A is absent. The upper left entry is the number of times the two genes co-occur in a library, and the middle right entry is the number of times neither gene occurs in a library. The off diagonal entries are the number of times one gene occurs, and the other does not. Both A and B are present eight times and absent 18 times. Gene A is present, and gene B is absent, two times; and gene B is present, and gene A is absent, two times. The probability (“p-value”) that the above association occurs due to chance as calculated using a Fisher exact test is 0.0003. Associations are generally considered significant if a p-value is less than 0.01 or 1.0e-2 (Agresti, supra; Rice, supra).

This method of estimating the probability for coexpression of two genes makes several assumptions. The method assumes that the libraries are independent and are identically sampled. However, in practical situations, the selected cDNA libraries are not entirely independent, because more than one library may be obtained from a single subject or tissue. Nor are they entirely identically sampled, because different numbers of cDNAs may be sequenced from each library. The number of cDNAs sequenced typically ranges from 5,000 to 10,000 cDNAs per library. In addition, because a Fisher exact coexpression probability is calculated for each gene versus 37,071 other assembled genes that occur in at least five libraries, a Bonferroni correction for multiple statistical tests is used.

Using the method, we have identified cDNAs that exhibit strong association, or coexpression, with known placental steroid synthesis genes, specifically aromatase P-450, cholesterol side-chain cleavage enzyme, meltrin-L, placental lactogen hormone, pregnancy-associated plasma protein-A, hydroxysteroid dehydrogenase, pregnancy-specific beta-glycoprotein, and placental alkaline phosphatase. Example V first shows the highly significant coexpression among the known genes and then among the novel cDNAs and the known placental steroid synthesis genes. The nine, novel cDNAs are characterized in Table 1 and can be used in place of the known genes, as surrogate markers, in the diagnosis, prognosis, evaluation of therapies or treatment of disorders associated with steroid-responsive tissues and with pregnancy. Further, the proteins or peptides expressed from the novel cDNAs are either potential therapeutics or targets for the identification or development of therapeutics.

Therefore, in one embodiment, the present invention encompasses a combination of cDNAs comprising the nucleic acid sequences of SEQ ID NOs:1-9. These nine cDNAs are shown by GBA and by transcript imaging to have strong coexpression with known placental steroid synthesis genes and with each other. The invention also provides a cDNA, its complement, and the use of a probe comprising the cDNA selected from SEQ ID NOs:1-9. The invention further provides SEQ ID NOs:4, 6, and 7 which encode a protein or a portion thereof.

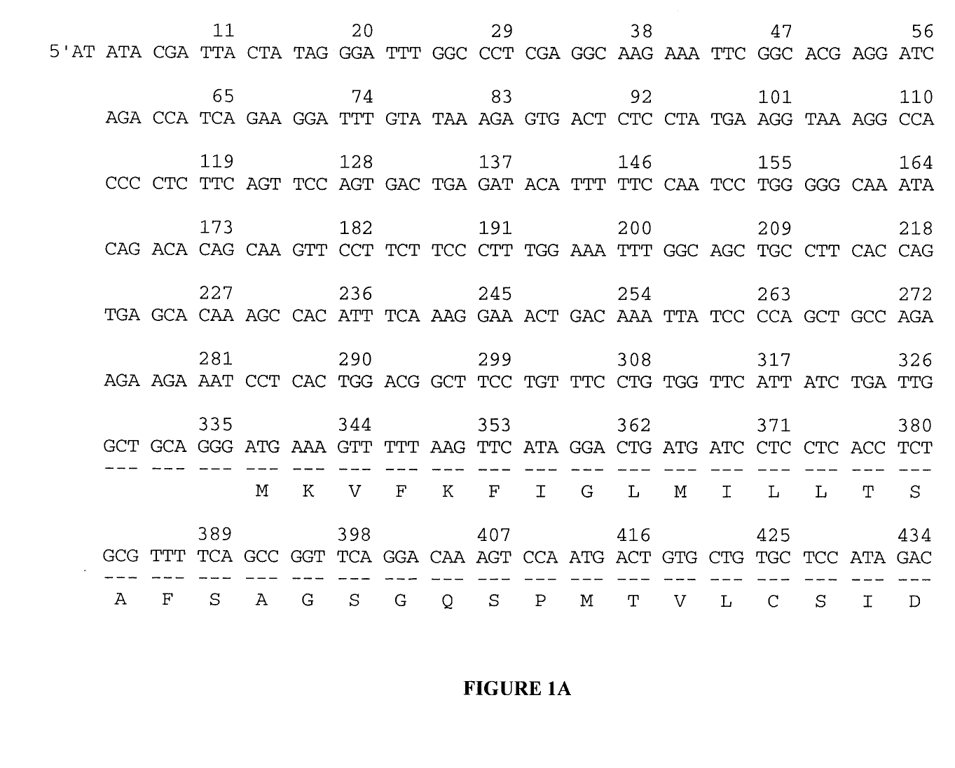

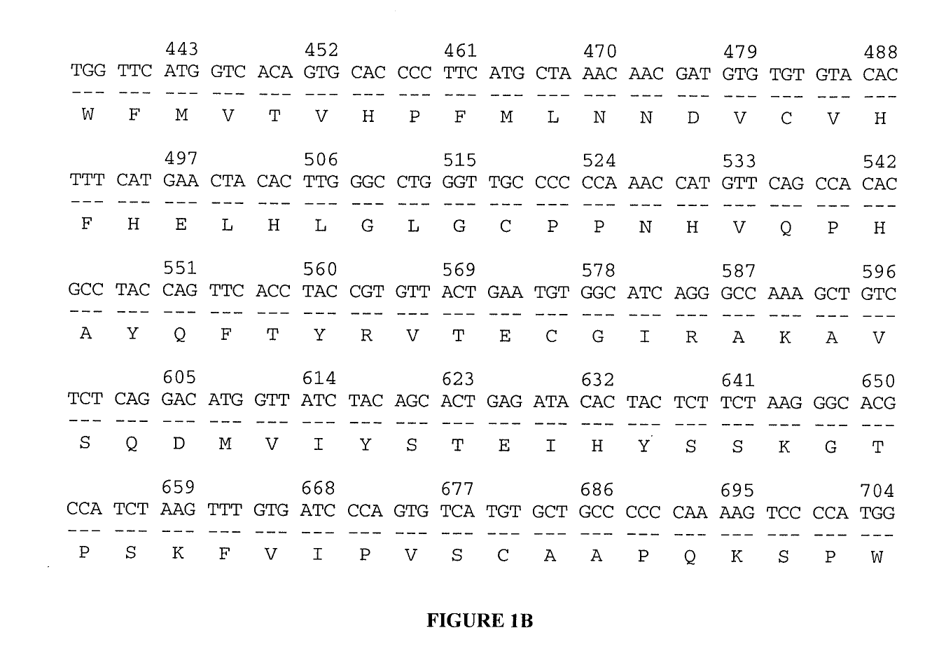

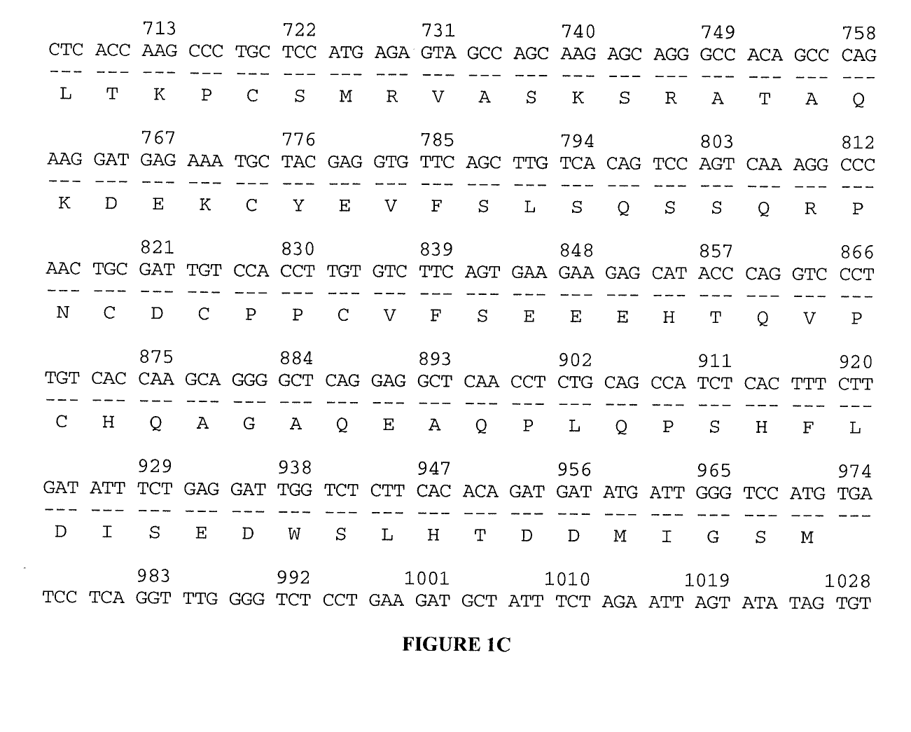

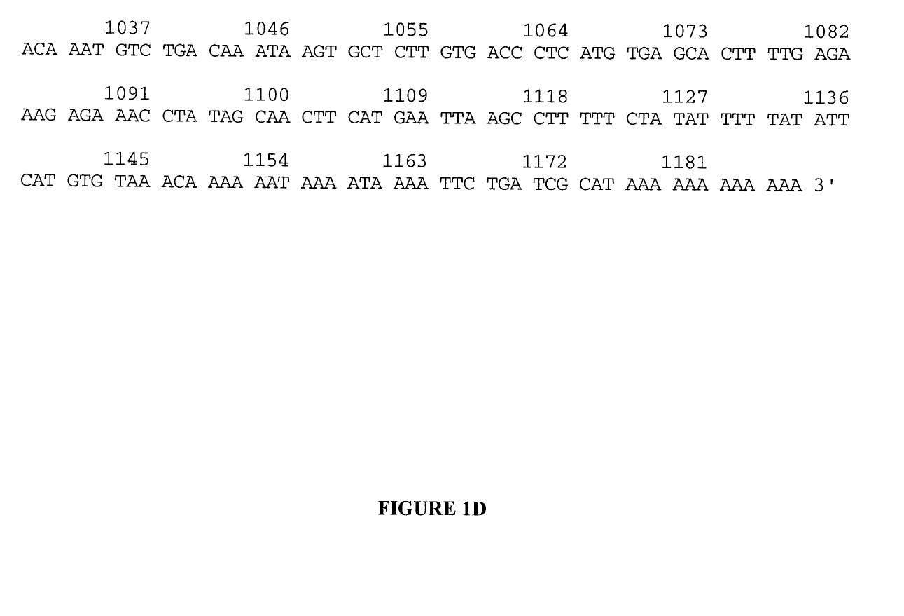

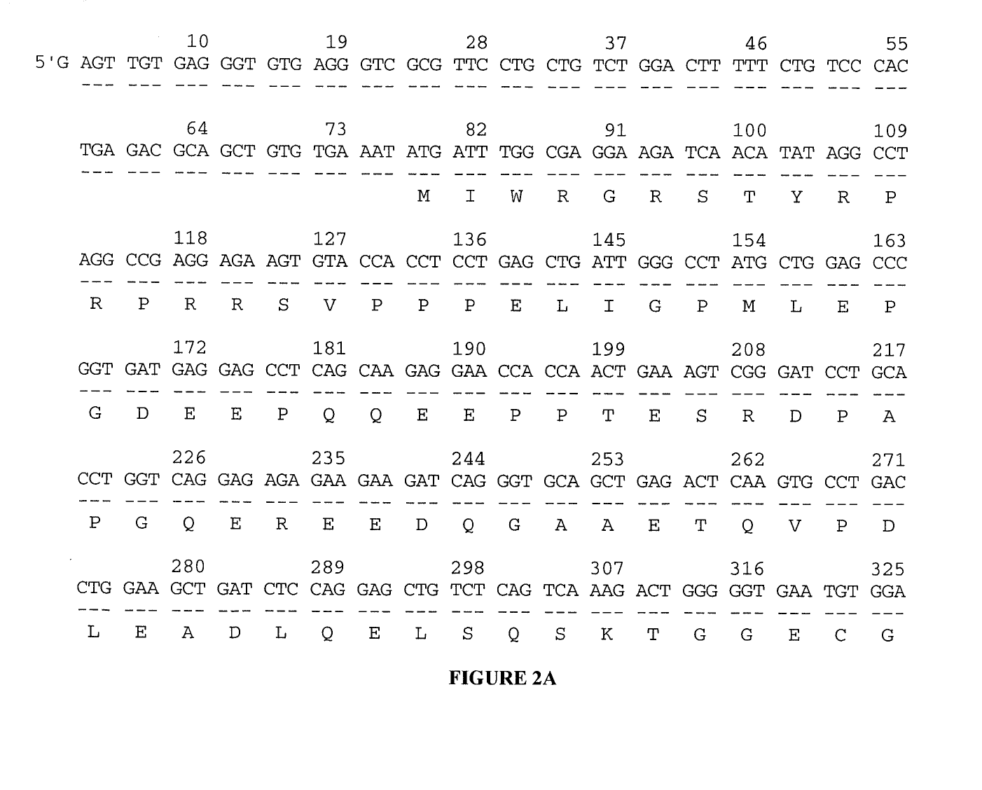

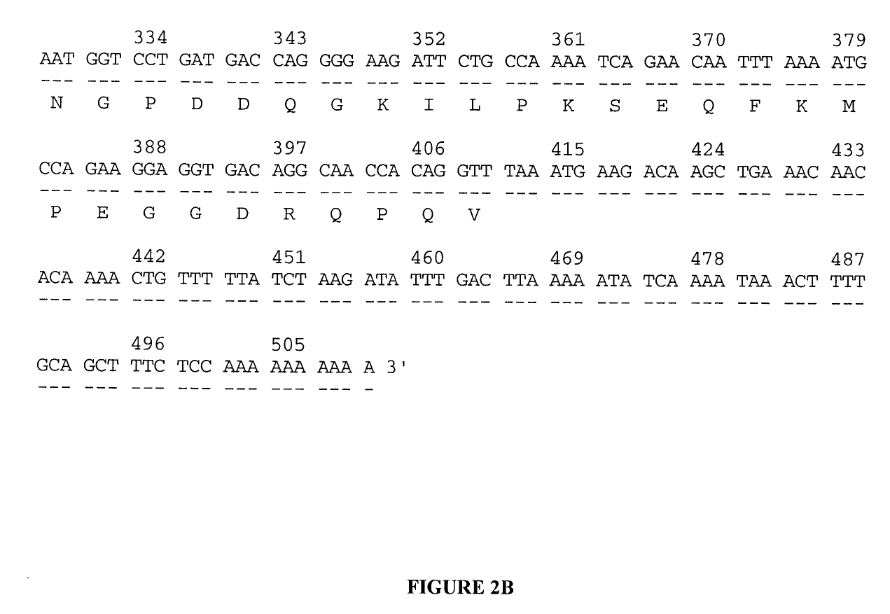

The invention further provides a purified protein encoded by SEQ ID NO:4 (FIGS. 1A-1D), 6, or 7 (FIGS. 2A and 2B) that is coexpressed with one or more known placental steroid synthesis genes in a plurality of biological samples.

The cDNA or the encoded protein may be used to search against the GenBank primate (pri), rodent (rod), mammalian (mam), vertebrate (vrtp), and eukaryote (eukp) databases, SwissProt, BLOCKS (Bairoch et al. (1997) Nucleic Acids Res 25:217-221), PFAM, and other databases that contain previously identified and annotated motifs, sequences, and gene functions. Methods that search for primary sequence patterns with secondary structure gap penalties (Smith et al. (1992) Protein Engineering 5:35-51) as well as algorithms such as Basic Local Alignment Search Tool (BLAST; Altschul (1993) J Mol Evol 36:290-300; Altschul et al. (1990) J Mol Biol 215:403-410), BLOCKS (Henikoff and Henikoff (1991) Nucleic Acids Res 19:6565-6572), Hidden Markov Models (HMM; Eddy (1996) Cur Opin Str Biol 6:361-365; Sonnhammer et al. (1997) Proteins 28:405-420), and the like, can be used to manipulate and analyze nucleotide and amino acid sequences. These databases, algorithms and other methods are well known in the art and are described in Ausubel et al. (1997; Short Protocols in Molecular Biology, John Wiley & Sons, New York N.Y., unit 7.7) and in Meyers (1995; Molecular Biology and Biotechnology, Wiley VCH, New York N.Y., p 856-853).

Also encompassed by the invention are polynucleotides that are capable of hybridizing to SEQ ID NOs:1-9, and fragments thereof under stringent conditions. Stringent conditions can be defined by salt concentration, temperature, and other chemicals and conditions well known in the art. Conditions can be selected, for example, by varying the concentrations of salt in the prehybridization, hybridization, and wash solutions or by varying the hybridization and wash temperatures. With some substrates, the temperature can be decreased by adding formamide to the prehybridization and hybridization solutions.

Hybridization can be performed at low stringency, with buffers such as 5×SSC (sodium saline citrate) with 1% sodium dodecyl sulfate (SDS) at 60° C., which permits complex formation between two nucleic acid sequences that contain some mismatches. Subsequent washes are performed at higher stringency with buffers such as 0.2×SSC with 0.1% SDS at either 45° C. (medium stringency) or 68° C. (high stringency), to maintain hybridization of only those complexes that contain completely complementary sequences. Background signals can be reduced by the use of detergents such as SDS, sarcosyl, or TRITON X-100 (Sigma-Aldrich, St. Louis Mo.), and/or a blocking agent, such as salmon sperm DNA. Hybridization methods are described in detail in Ausubel (supra, units 2.8-2.11, 3.18-3.19 and 4-6-4.9) and Sambrook et al. (1989; Molecular Cloning, A Laboratory Manual, Cold Spring Harbor Press, Plainview N.Y.)

A cDNA can be extended utilizing a partial nucleotide sequence and employing various PCR-based methods known in the art to detect upstream sequences such as promoters and other regulatory elements. (See, e.g., Dieffenbach and Dveksler (1995) PCR Primer, a Laboratory Manual, Cold Spring Harbor Press, Plainview N.Y.). Additionally, one may use an XL-PCR kit (Applied Biosystems (ABI), Foster City Calif.), nested primers, and commercially available cDNA libraries (Clontech, Palo Alto Calif.) or genomic libraries (Clontech) to extend the sequence. For all PCR-based methods, primers may be designed using LASERGENE software, DNASTAR, Madison Wis.) or other commercially available software, to be about 15 to 30 nucleotides in length, to have a GC content of about 50%, and to form a hybridization complex at temperatures of about 68° C. to 72° C.

In another aspect of the invention, the cDNA can be cloned into a recombinant vector that directs the expression of the protein, or structural or functional portions thereof, in host cells. Due to the inherent degeneracy of the genetic code, other DNA sequences which encode substantially the same or a functionally equivalent amino acid sequence may be produced and used to express the protein encoded by the cDNA. The nucleotide sequences of the present invention can be engineered using methods generally known in the art in order to alter the nucleotide sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

In order to express a biologically active protein, the cDNA or derivatives thereof, may be inserted into an expression vector, i.e., a vector which contains the elements for transcriptional and translational control of the inserted coding sequence in a particular host. These elements include regulatory sequences, such as enhancers, constitutive and inducible promoters, and 5′ and 3′ untranslated regions. Methods which are well known to those skilled in the art may be used to construct such expression vectors. These methods include in vitro recombinant DNA techniques, synthetic techniques, and in vivo genetic recombination (Sambrook, supra; Ausubel, supra).

A variety of expression vector/host cell systems may be utilized to express the cDNA. These include, but are not limited to, microorganisms such as bacteria transformed with recombinant bacteriophage, plasmid, or cosmid expression vectors; yeast transformed with yeast expression vectors; insect cell systems infected with baculovirus vectors; plant cell systems transformed with viral or bacterial expression vectors; or animal cell systems. For long term production of recombinant proteins in mammalian systems, stable expression in cell lines is preferred. For example, the cDNA can be transformed into cell lines using expression vectors which may contain viral origins of replication and/or endogenous expression elements and a selectable or visible marker gene on the same or on a separate vector. The invention is not to be limited by the vector or host cell employed.

In general, host cells that contain the cDNA and that express the protein may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or amino acid sequences. Immunological methods for detecting and measuring the expression of the protein using either specific polyclonal or monoclonal antibodies are known in the art. Examples of such techniques include enzyme-linked immunosorbent assays (ELISAs), radioimmunoassays (RIAs), and fluorescence activated cell sorting (FACS).

Host cells transformed with the cDNA may be cultured under conditions for the expression and recovery of the protein from cell culture. The protein produced by a transgenic cell may be secreted or retained intracellularly depending on the sequence and/or the vector used. As will be understood by those of skill in the art, expression vectors containing the cDNA may be designed to contain signal sequences which direct secretion of the protein through a prokaryotic or eukaryotic cell membrane.

In addition, a host cell strain may be chosen for its ability to modulate expression of the inserted sequences or to process the expressed protein in the desired fashion. Such modifications of the protein include, but are not limited to, acetylation, carboxylation, glycosylation, phosphorylation, lipidation, and acylation. Post-translational processing which cleaves a “prepro” form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the ATCC (Manassas Va.) and may be chosen to ensure the correct modification and processing of the expressed protein.

In another embodiment of the invention, natural, modified, or recombinant nucleic acid sequences are ligated to a heterologous sequence resulting in translation of a fusion protein containing heterologous protein moieties in any of the aforementioned host systems. Such heterologous protein moieties facilitate purification of fusion proteins using commercially available affinity matrices. Such moieties include, but are not limited to, glutathione S-transferase, maltose binding protein, thioredoxin, calmodulin binding peptide, 6-His, FLAG, c-myc, hemaglutinin, and monoclonal antibody epitopes.

In another embodiment, the cDNAs, wholly or in part, are synthesized using chemical or enzymatic methods well known in the art (Caruthers et al. (1980) Nucl Acids Symp Ser (7) 215-233; Ausubel, supra). For example, peptide synthesis can be performed using various solid-phase techniques (Roberge et al. (1995) Science 269:202-204), and machines such as the ABI 431A peptide synthesizer (ABI) can be used to automate synthesis. If desired, the amino acid sequence may be altered during synthesis and/or combined with sequences from other proteins to produce a variant.

Screening, Diagnostics and Therapeutics

The cDNAs can be used in diagnosis, prognosis, selection and evaluation of therapies and treatment of disorders associated with steroid-responsive tissues and with pregnancy including, but not limited to, PIH, preeclampsia, hyperinsulinemia, glucose intolerance, insulin insensitivity, fetal growth retardation, fetal Down syndrome and estrogen-sensitive adenocarcinomas of the breast, ovary and uterus.

The cDNAs may be used to screen a plurality of molecules for specific binding affinity. The assay can be used to screen a plurality of DNA molecules, RNA molecules, peptide nucleic acids (PNAs), peptides, ribozymes, antibodies, agonists, antagonists, immunoglobulins, inhibitors, proteins including transcription factors, enhancers, repressors, and drugs and the like which regulate the activity of the polynucleotide in the biological system. The assay involves providing a plurality of molecules, combining the cDNA or a fragment thereof with the plurality of molecules under conditions to allow specific binding, and detecting specific binding to identify at least one molecule which specifically binds the cDNA.

Similarly the proteins or portions thereof may be used to screen libraries of molecules or compounds in any of a variety of screening assays. The portion of a protein employed in such screening may be free in solution, affixed to an abiotic or biotic substrate (e.g. borne on a cell surface), or located intracellularly. Specific binding between the protein and the molecule may be measured. The assay can be used to screen a plurality of DNA molecules, RNA molecules, PNAs, peptides, mimetics, ribozymes, antibodies, agonists, antagonists, immunoglobulins, inhibitors, peptides, polypeptides, drugs and the like, which specifically bind the protein. One method for high throughput screening using very small assay volumes and very small amounts of test compound is described in Burbaum et al. U.S. Pat. No. 5,876,946, incorporated herein by reference, which screens large numbers of molecules for enzyme inhibition or receptor binding.

In one preferred embodiment, the cDNAs are used for diagnostic purposes to determine the absence, presence, or altered—increased or decreased compared to a normal standard—expression of the gene. The polynucleotide consists of complementary RNA and DNA molecules, branched nucleic acids, and/or PNAs. In one alternative, the polynucleotides are used to detect and quantify gene expression in samples in which expression of the cDNA is correlated with disease. In another alternative, the cDNA can be used to detect genetic polymorphisms associated with a disease. These polymorphisms may be detected in the transcript cDNA.

The specificity of the probe is determined by whether it is made from a unique region, a regulatory region, or from a conserved motif. Both probe specificity and the stringency of diagnostic hybridization or amplification (maximal, high, intermediate, or low) will determine whether the probe identifies only naturally occurring, exactly complementary sequences, allelic variants, or related sequences. Probes designed to detect related sequences should preferably have at least 50% sequence identity to any of the polynucleotides encoding the protein.

Methods for producing hybridization probes include the cloning of nucleic acid sequences into vectors for the production of MRNA probes. Such vectors are known in the art, are commercially available, and may be used to synthesize RNA probes in vitro by adding RNA polymerases and labeled nucleotides. Hybridization probes may incorporate nucleotides labeled by a variety of reporter groups including, but not limited to, radionuclides such as ³²p or ³⁵S, enzymatic labels such as alkaline phosphatase coupled to the probe via avidin/biotin coupling systems, fluorescent labels, and the like. The labeled cDNAs may be used in Southern or northern analysis, dot blot, or other membrane-based technologies; in PCR technologies; and in microarrays utilizing samples from subjects to detect altered protein expression.

The cDNA can be labeled by standard methods and added to a sample from a subject under conditions for the formation and detection of hybridization complexes. After incubation the sample is washed, and the signal associated with hybrid complex formation is quantitated and compared with a standard value. Standard values are derived from any control sample, typically one that is free of the suspect disease. If the amount of signal in the subject sample is altered in comparison to the standard value, then the presence of altered levels of expression in the sample indicates the presence of the disease. Qualitative and quantitative methods for comparing the hybridization complexes formed in subject samples with previously established standards are well known in the art.

Such assays may also be used to evaluate the efficacy of a particular therapeutic treatment regimen in animal studies, in clinical trials, or to monitor the treatment of an individual subject. Once the presence of disease is established and a treatment protocol is initiated, hybridization or amplification assays can be repeated on a regular basis to determine if the level of expression in the patient begins to approximate that which is observed in a healthy subject. The results obtained from successive assays may be used to show the efficacy of treatment over a period ranging from several days to many years.

The cDNAs may be used for the diagnosis of a variety of disorders associated with steroid-responsive tissues and with pregnancy. These include, but are not limited to, PIH, preeclampsia, hyperinsulinemia, glucose intolerance, insulin insensitivity, fetal growth retardation, Down syndrome pregnancy, and estrogen-sensitive adenocarcinomas of the breast, ovary and uterus.

In yet another alternative, polynucleotides may be used to generate hybridization probes useful in mapping the naturally occurring genomic sequence. Fluorescent in situ hybridization (FISH) may be correlated with other physical chromosome mapping techniques and genetic map data as described in Heinz-Ulrich et al. (In: Meyers, supra, pp. 965-968).

Arrays incorporating cDNAs, proteins, or antibodies may be prepared and analyzed using methods well known in the art. Oligonucleotides or cDNAs may be used as hybridization probes or targets to monitor the expression level of large numbers of genes simultaneously or to identify genetic variants, mutations, and single nucleotide polymorphisms. Proteins may be used to identify ligands, to investigate protein:protein interactions, or to produce a proteomic profile of gene expression (i.e., to detect and quantify expression of a protein in a sample). Antibodies may be also be used produce a proteomic profile of gene expression. Such arrays may be used to determine gene function; to understand the genetic basis of a condition, disease, or disorder; to diagnose a condition, disease, or disorder; and to develop and monitor the activities of therapeutic agents. (See, e.g., Brennan et al. (1995) U.S. Pat. No. 5,474,796; Schena et al. (1996) Proc Natl Acad Sci 93:10614-10619; Heller et al. (1997) Proc Natl Acad Sci 94:2150-2155; Heller et al. (1997) U.S. Pat. No. 5,605,662; and deWildt et al. (2000) Nature Biotechnol 18:989-994.)

In another embodiment, antibodies or Fabs comprising an antigen binding site that specifically binds the protein may be used for the diagnosis of disorders associated with steroid-responsive tissues and with pregnancy. and characterized by the over-or-under expression of the protein. A variety of protocols for measuring protein expression, including ELISAs, RIAs, and FACS, are well known in the art and provide a basis for diagnosing altered or abnormal levels of expression. Standard values for protein expression are established by combining samples taken from healthy subjects, preferably human, with antibody to the protein under conditions for complex formation. The amount of complex formation may be quantitated by various methods, preferably by photometric means. Quantities of the protein expressed in disease samples are compared with standard values. Deviation between standard and subject values establishes the parameters for diagnosing or monitoring disease. Alternatively, one may use competitive drug screening assays in which neutralizing antibodies capable of binding specifically with the protein compete with a test compound. Antibodies can be used to detect the presence of any peptide which shares one or more antigenic determinants with the protein. In one aspect, the antibodies of the present invention can be used for treatment or monitoring therapeutic treatment for disorders associated with steroid-responsive tissues and with pregnancy.

In another aspect, the cDNA, or its complement, may be used therapeutically for the purpose of expressing mRNA and protein, or conversely to block transcription or translation of the mRNA. Expression vectors may be constructed using elements from retroviruses, adenoviruses, herpes or vaccinia viruses, or bacterial plasmids, and the like. These vectors may be used for delivery of nucleotide sequences to a particular target organ, tissue, or cell population. Methods well known to those skilled in the art can be used to construct vectors to express nucleic acid sequences or their complements. (See, e.g., Maulik et al. (1997) Molecular Biotechnology, Therapeutic Applications and Strategies, Wiley-Liss, New York N.Y.) Alternatively, the cDNA or its complement, may be used for somatic cell or stem cell gene therapy. Vectors may be introduced in vivo, in vitro, and ex vivo. For ex vivo therapy, vectors are introduced into stem cells taken from the subject, and the resulting transgenic cells are clonally propagated for autologous transplant back into that same subject. Delivery of the cDNA by transfection, liposome injections, or polycationic amino polymers may be achieved using methods which are well known in the art. (See, e.g., Goldman et al. (1997) Nature Biotechnology 15:462-466.) Additionally, endogenous gene expression may be inactivated using homologous recombination methods which insert an inactive gene sequence into the coding region or other targeted region of the cDNA. (See, e.g. Thomas et al. (1987) Cell 51: 503-512.)

Vectors containing the cDNA can be transformed into a cell or tissue to express a missing protein or to replace a nonfunctional protein. Similarly a vector constructed to express the complement of the cDNA can be transformed into a cell to downregulate the protein expression. Complementary or antisense sequences may consist of an oligonucleotide derived from the transcription initiation site; nucleotides between about positions −10 and +10 from the ATG are preferred. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee et al. In: Huber and Carr (1994) Molecular and Immunologic Approaches, Futura Publishing, Mt. Kisco N.Y., pp. 163-177.)

Ribozymes, enzymatic RNA molecules, may also be used to catalyze the cleavage of mRNA and decrease the levels of particular mRNAs, such as those comprising the cDNAs of the invention. (See, e.g., Rossi (1994) Current Biology 4: 469-471.) Ribozymes may cleave mRNA at specific cleavage sites. Alternatively, ribozymes may cleave mRNAs at locations dictated by flanking regions that form complementary base pairs with the target MRNA. The construction and production of ribozymes is well known in the art and is described in Meyers (supra).

RNA molecules may be modified to increase intracellular stability and half-life. Possible modifications include, but are not limited to, the addition of flanking sequences at the 5′ and/or 3′ ends of the molecule, or the use of phosphorothioate or 2′O-methyl rather than phosphodiester linkages within the backbone of the molecule. Alternatively, nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases, may be included.

Further, an antagonist, or an antibody that binds specifically to the protein may be administered to a subject to treat a disorder associated with pregnancy. The antagonist, antibody, or fragment may be used directly to inhibit the activity of the protein or indirectly to deliver a therapeutic agent to cells or tissues which express the protein. The therapeutic agent may be a cytotoxic agent selected from a group including, but not limited to, abrin, ricin, doxorubicin, daunorubicin, taxol, ethidium bromide, mitomycin, etoposide, tenoposide, vincristine, vinblastine, colchicine, dihydroxy anthracin dione, actinomycin D, diphteria toxin, Pseudomonas exotoxin A and 40, radioisotopes, and glucocorticoid.

Antibodies to the protein may be generated using methods that are well known in the art. Such antibodies may include, but are not limited to, polyclonal, monoclonal, chimeric, and single chain antibodies, Fab fragments, and fragments produced by a Fab expression library. Neutralizing antibodies, such as those which inhibit dimer formation, are especially preferred for therapeutic use. Monoclonal antibodies to the protein may be prepared using any technique which provides for the production of antibody molecules by continuous cell lines in culture. These include, but are not limited to, the hybridoma, the human B-cell hybridoma, and the EBV-hybridoma techniques. In addition, techniques developed for the production of chimeric antibodies can be used. (See, e.g., Pound (1998) Immunochemical Protocols, Methods Mol Biol Vol. 80). Alternatively, techniques described for the production of single chain antibodies may be employed. Fabs which contain specific binding sites for the protein may also be generated. Various immunoassays may be used to identify antibodies having the desired specificity. Numerous protocols for competitive binding or immunoradiometric assays using either polyclonal or monoclonal antibodies with established specificities are well known in the art.

Yet further, an agonist of the protein may be administered to a subject to treat or prevent a disease associated with decreased expression, longevity or activity of the protein.

An additional aspect of the invention relates to the administration of a pharmaceutical or sterile composition, in conjunction with a pharmaceutically acceptable carrier, for any of the therapeutic applications discussed above. Such pharmaceutical compositions may consist of the protein or antibodies, mimetics, agonists, antagonists, or inhibitors of the protein. The compositions may be administered alone or in combination with at least one other agent, such as a stabilizing compound, which may be administered in any sterile, biocompatible pharmaceutical carrier including, but not limited to, saline, buffered saline, dextrose, and water. The compositions may be administered to a subject alone or in combination with other agents, drugs, or hormones.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Mack Publishing, Easton Pa.).

For any compound, the therapeutically effective dose can be estimated initially either in cell culture assays or in animal models such as mice, rats, rabbits, dogs, or pigs. An animal model may also be used to determine the concentration range and route of administration. Such information can then be used to determine useful doses and routes for administration in humans.

A therapeutically effective dose refers to that amount of active ingredient which ameliorates the symptoms or condition. Therapeutic efficacy and toxicity may be determined by standard pharmaceutical procedures in cell cultures or with experimental animals, such as by calculating and contrasting the ED₅₀ (the dose therapeutically effective in 50% of the population) and LD₅₀ (the dose lethal to 50% of the population) statistics. Any of the therapeutic compositions described above may be applied to any subject in need of such therapy, including, but not limited to, mammals such as dogs, cats, cows, horses, rabbits, monkeys, and most preferably, humans.

### EXAMPLES

I cDNA Library Construction

The PLACNOT02 cDNA library was constructed from microscopically normal placenta tissue removed along with a 21-week-old Hispanic female fetus (specimen #RB95-06-0376) who was delivered following fetal demise. The mother had a history of miscarriages.

The frozen tissue was homogenized and lysed in TRIZOL reagent (1 g tissue/10 ml; Invitrogen) using a POLYTRON homogenizer (Brinkmann Instruments, Westbury N.Y.) in guanidinium isothiocyanate solution. The lysate was centrifuged over a 5.7 M CsCl cushion using an SW28 rotor in an L8-70M ultracentrifuge (Beckman Coulter, Fullerton Calif.) for 18 hours at 25,000 rpm at ambient temperature. The RNA was extracted with acid phenol, pH 4.7, precipitated using 0.3 M sodium acetate and 2.5 volumes of ethanol, resuspended in RNAse-free water, and treated with DNAse at 37° C. The RNA extraction and precipitation were repeated once. The mRNA was isolated with the OLIGOTEX kit (Qiagen, Chatsworth Calif.) and used to construct the cDNA library.

The mRNA was handled according to the recommended protocols in the SUPERSCRIPT plasmid system (Invitrogen). The cDNAs were fractionated on a SEPHAROSE CL4B column (Amersham Pharmacia Biotech (APB), Piscataway N.J.), and those cDNAs exceeding 400 bp were ligated into pINCY plasmid (Incyte Genomics, Palo Alto Calif.). The plasmid was subsequently transformed into DH5α competent cells (Invitrogen). Other libraries from the Embryonic Structures category of LIFESEQ Gold database (Incyte Genomics) are listed below. Procedures similar to those just described were used to prepare the cDNAs in each of the following embryo, fetal, or placental libraries.

| Library | cDNAs | Description of Tissue |
|---|---|---|
| EMBRFEP01 | 2313 | embryo, 8 w, TIGR |
| EMBRFEP02 | 680 | embryo, 6 w, TIGR |
| FETAFEM01 | 20779 | fetus, 8-9 w, pool, NORM, CGAP/WM/WN |
| FETAFEP01 | 1641 | fetus, 12 w, TIGR |
| FETAFEP02 | 1176 | fetus, 12 w, TIGR |
| FETAFEP03 | 1366 | fetus, 9 w, TIGR |
| PLACFEB01 | 5869 | placenta, aw/hydrocephalus, fetal demise, |
| | | 16 w, 15 wM, pool |
| PLACFEC01 | 693 | placenta, aw/hydrocephalus, fetal, 16 w, |
| | | lg cDNA |
| PLACFEP01 | 1251 | placenta, fetal, 3′ TIGR |
| PLACFEP02 | 649 | placenta, fetal, TIGR |
| PLACFER01 | 6579 | placenta, aw/hydrocephalus, fetal, 16 w, RP |
| PLACFER06 | 7229 | placenta, aw/hydrocephalus, fetal, 16 w, 5 RP |
| PLACFET04 | 3604 | placenta, fetal, 18 wM |
| PLAGNOB01 | 3954 | placenta, neonatal, F |
| PLACNOM01 | 1935 | placenta, fetal, M, WM |
| PLACNOM02 | 18495 | placenta, neonatal, F, NORM, WM |
| PLACNOM03 | 12091 | placenta, fetal, 8-9 w, pool, NORM, |
| | | CGAP/WM |
| PLACNOR01 | 4357 | placenta, aw/hydrocephalus, fetal, |
| | | 16 w/18 wM, pool, RP |
| PLACNOT02 | 5917 | placenta, fetal, 21 wF |
| PLACNOT05 | 3461 | placenta, fetal, 18 wM |
| PLACNOT07 | 2674 | placenta, aw/hydrocephalus, fetal, 16 w |

II Construction of pINCY Plasmid

The plasmid was constructed by digesting the pSPORT1 plasmid (Invitrogen) with EcoRI restriction enzyme (New England Biolabs, Beverly Mass.) and filling the overhanging ends using Klenow enzyme (New England Biolabs) and 2′-deoxynucleotide 5′-triphosphates (dNTPs). The plasmid was self-ligated and transformed into the bacterial host, E. coli strain JM109.

An intermediate phasmid produced by the bacteria (pSPORT 1-ΔRI) showed no digestion with EcoRI and was digested with Hind III (New England Biolabs) and the overhanging ends were again filled in with Klenow and dNTPs. A linker sequence was phosphorylated, ligated onto the 5′ blunt end, digested with EcoRI, and self-ligated. Following transformation into JM109 host cells, plasmids were isolated and tested for preferential digestibility with EcoRI, but not with Hind m. A single colony that met this criteria contained the pINCY plasmid.

After testing the plasmid for its ability to incorporate cDNAs from a library prepared using NotI and EcoRI restriction enzymes, several clones were sequenced; and a single clone containing an insert of approximately 0.8 kb was selected from which to prepare a large quantity of the plasmid. After digestion with NotI and EcoRI, the plasmid was isolated on an agarose gel and purified using a QIAQUICK column (Qiagen) for use in library construction.

III Isolation and Sequencing of cDNA Clones

Plasmid DNA was released from the cells and purified using the REAL PREP 96 plasmid kit (Qiagen). The recommended protocol was employed except for the following changes: 1) the bacteria were cultured in 1 ml of sterile TERRIFIC BROTH (BD Biosciences, Sparks Md.) with carbenicillin (carb) at 25 mg/l and glycerol at 0.4%; 2) the cultures were incubated for 19 hours after the wells were inoculation and then lysed with 0.3 ml of lysis buffer; 3) following isopropanol precipitation, the DNA pellet was resuspended in 0.1 ml of distilled water. After the last step in the protocol, samples were transferred to a 96-well block for storage at 4° C.

The cDNAs were prepared using a MICROLAB 2200 system (Hamilton, Reno Nev.) in combination with DNA ENGINE thermal cyclers (MJ Research, Watertown Mass.). The cDNAs were sequenced by the method of Sanger and Coulson (1975; J Mol Biol 94:441f) using ABI PRISM 377 DNA sequencing systems (ABI). Most of the sequences were sequenced using standard solutions, dyes, protocols and kits (ABI; APB); in some cases, solution volumes were reduced to 0.25×-1.0×.

IV Selection, Assembly, and Characterization of Sequences

The sequences used for co-expression analysis represent full length coding sequences or were assembled from EST sequences, 5′ and 3′ long read sequences, and full length coding sequences.

The assembly process is described as follows. EST sequence chromatograms were processed and verified. Quality scores were obtained using PHRED (Ewing et al. (1998) Genome Res 8:175-185; Ewing and Green (1998) Genome Res 8:186-194), and edited sequences were loaded into a relational database management system (RDBMS). The sequences were clustered using BLAST with a product score of 50. All clusters of two or more sequences created a bin which represents one transcribed gene.

Assembly of the component sequences within each bin was performed using a modification of Phrap, a publicly available program for assembling DNA fragments (Green, P. University of Wash., Seattle Wash.). Bins that showed 82% identity from a local pair-wise alignment between any of the consensus sequences were merged.

Bins were annotated by screening the consensus sequence in each bin against public databases, such as GBpri and GenPept from NCBI. The annotation process involved a FASTn screen against the GBpri database in GenBank. Those hits with a percent identity of greater than or equal to 75% and an alignment length of greater than or equal to 100 base pairs were recorded as homolog hits. The residual unannotated sequences were screened by FASTx against GenPept. Those hits with an E value of less than or equal to 10⁻⁸ were recorded as homolog hits.

Sequences were then reclustered using BLASTn and Cross-Match, a program for rapid amino acid and nucleic acid sequence comparison and database search (Green, supra), sequentially. Any BLAST alignment between a sequence and a consensus sequence with a score greater than 150 was realigned using cross-match. The sequence was added to the bin whose consensus sequence gave the highest Smith-Waterman score (Smith et al. (1992) Protein Engineering 5:35-51) amongst local alignments with at least 82% identity. Non-matching sequences were moved into new bins, and assembly processes were repeated.

V Coexpression Analyses of Known Placental Steroid Synthesis Genes

Eight genes known to be involved in steroid synthesis or metabolism and with disorders associated with steroid-responsive tissues and with pregnancy were selected to identify coexpressing novel cDNAs. Each of the known genes is briefly described.:

Aromatase P-450 (P-450arom)

P-450arom catalyzes estrogen 2-hydroxylase activity in human placenta and catalyzes the conversion of testosterone to estradiol. Total P-450arom in the placenta increases as pregnancy progresses, and contributes to the marked increase in maternal estrogen production. Altered synthesis of steroids is significant because of their role in pregnancy-induced hypertension (PIH). For example, mutations in the steroid receptor S810L cause PIH by altering the response to progesterone elevation in pregnancy and increasing salt and water retention. The effect of catechol estrogen on PIH may be mediated by human placental steroidogenesis; estrogen 2-hydroxylase is significantly higher in PIH placenta than in normal placenta. Cigarette smoking during pregnancy lowers aromatase cytochrome p-450 in the placenta and causes an alteration in placental estrogen producing ability. Insulin and the insulin-like growth factors regulate placental steroidogenesis by modulating the activity of placental aromatase P450 and other placental steroid synthesis enzymes (Nestler (1987, 1989, 1991, and 1993; supra); Toda et al. (1989) FEBS Lett 247:371-6; Inoue et al. (1992) Nippon Sanka Fujinka Gakkai Zasshi 44:581-8; Kitawaki et al. (1993) J Steroid Biochem Mol Biol 45:485-91; Kitawaki et al. (1992) Endocrinology 130:2751-7; and Okubo et al. (1996) Endocr J 43:363-8).

Cholesterol Side-Chain Cleavage Enzyme (P450scc)

P450scc catalyzes the first and key regulatory reaction controlling steroid hormone synthesis. It is expressed in the placenta in early and midgestation. Concentrations of steroid-synthesis genes and prostaglandins, which regulate physiological functions in reproductive tissues, increase during pregnancy, and are dependent on P450scc. Insulin and the insulin-like growth factors regulate placental steroidogenesis by modulating the activity of P450scc, among other placental steroid synthesis enzymes (Turcan et al. (1981) Biochem Pharmacol 30:1223-5; Chung et al. (1986) Proc Natl Acad Sci 83:8962-6; Nestler (1987, 1989, 1991, and 1993, supra); Schiff et al. (1993) Endocrinology 133:529-37; Campisi et al. (1994) Acta Eur Fertil 25:295-7; Hakkola et al. (1996) Biochem Pharmacol 52:379-83; Okita and Okita (1996) Crit Rev Biochem Mol Biol 31:101-26; and Albrecht and Daels (1997) J Reprod Fertil 111:127-33).

Hydroxysteroid Dehydrogenase: 3-beta-hydroxysteroid Dehydrogenase (3-beta HSD)

3-beta HSD catalyzes oxidation of beta-hydroxysteroid precursors, leading to the synthesis of all classes of steroid hormones. Several isoenzymes are known, including a placentally-expressed 3-beta HSD isoenzyme. Insulin and the insulin-like growth factors regulate placental steroidogenesis by modulating the activity of 3 beta-HSD, among other placental steroid synthesis enzymes. (Neto et al. (1979) Acta Paediatr Scand 68:459-64; Nestler (1987, 1989, 1991 and 1993, supra) Couet et al. (1990) Endocrinology 127:2141-8; Dupont et al. (1990) Mol Cell Endocrinol 74:R7-10; Martel et al. (1990) Endocrinology 127:2726-37; Dupont et al. (1991) J Androl 12:161-4; Rheaume et al. (1991) Mol Endocrinol 5:1147-57; Zhao et al. (1991) J Biol Chem 266:583-93; Couet et al. (1992) Endocrinology 131:3034-44; Dupont et al. (1992) J Clin Endocrinol Metab 74:994-8; Bain (1993) Genomics 16:219-2; Juneau et al. (1993) Biol Reprod 48:226-34; Riley et al. (1993) Gynecol Obstet Invest 35:199-203; Juengel et al. (1994) Biol Reprod 51:380-4; Martel et al. (1994) Mol Cell Endocrinol 104:103-11).

Meltrin-L Precursor/a Disintegrin and Metalloprotease (ADAM-12)

A metalloprotease abundant in human term placenta as well as some tumor cell lines. ADAM-12 exists as an alternatively spliced soluble secreted protein. ADAM-12 cleaves IGFBP to release IGF. During pregnancy, ADAM-12 contributes to the IGFBP-3 protease activity present in pregnancy serum. (Gilpin et al. (1998) J Biol Chem 273:157-66; and Shi et al. (2000) J Biol Chem 275: 18574-80).

Placental Alkaline Phosphatase (PLAP)

A placental enzyme whose expression is regulated by estradiol, PLAP and its mRNA are found in placenta as early as 7 weeks of gestation and continue to increase throughout pregnancy. Determination of placental alkaline phosphatase is used in detecting the damages of alveolar type I cells caused by smoke inhalation. PLAP may play a role in feto-maternal metabolism and placental differentiation. The increase of serum PLAP may be helpful for the diagnosis of ovarian cancer (Kam et al. (1985) Proc Natl Acad Sci 82:8715-9; Okamoto, et al. (1990) Proc Natl Acad Sci 82:8715-9; Wang et al. (1996) Chung Hua Fu Chan Ko Tsa Chih 31:107-9; and Xie, supra).

Placental Lactogen Hormone (PL-4)/Chorionic Somatomammotropin

PL-4 and the human growth hormones (hGH) regulate maternal and fetal metabolism and the growth and development of the fetus. PL and hGH stimulate maternal production of insulin-like growth factor (IGF). In the fetus, PL stimulates the production of IGFs, insulin, and adrenocortical hormones. In women at risk for FGR, higher levels of PL are associated with a decreased prevalence of FGR (Gardner, supra; Handwerger and Freemark (2000) J Pediatr Endocrinol Metab 13:343-56).

Pregnancy-Associated Plasma Protein-A (PAPP-A)

PAPP-A is a metalloprotease expressed in the placenta that cleaves IGFBP to release bound IGF. PAPP-A is reduced in pregnancies with fetal Down Syndrome and is used to diagnose the disease. Levels of maternal serum PAPP-A are reduced in smokers by approximately 15 percent (Stabile et al. (1988) Obstet Gynecol Surv 43:73-82; Ruge et al. (1990) Acta Obstet Gynecol Scand 69:589-95; Kristensen et al. (1994) Biochemistry 33:1592-8; Bersinger et al. (1995) Reprod Fertil Dev 7:1419-23; Haaning et al. (1996) Eur J Biochem 237:159-63; Qin, supra; Morssink et al. (1998) Prenat Diagn 18:147-52; Bersinger et al. (1999) Prenat Diagn 19:537-41; De Biasio, supra; and Spencer, supra).

Pregnancy-Specific Beta-1-Glycoprotein (PS Beta G)

PS beta G, a major product of the placenta, consists of a set of glycoproteins synthesized by the syncytiotrophoblast. It may be able to predict the outcome of pregnancy with threatened abortion. An evolutionary relationship between CEA and PS beta G points to a possible common function in the control of cell invasion and/or metastasis. In women at risk for FGR, higher levels of PS beta G are associated with a decreased prevalence of FGR (Streydio et al. (1988) Biochem Biophys Res Commun 154:130-7; Watanabe and Chou (1988) Biochem Biophys Res Commun 152:762-8; Gardner, supra; and Jurisicova, supra).

Each known gene was scored as present in a library if at least one mRNA/cDNA was detected in the sample and as absent if no mRNA/cDNA was detected. The co-occurrences of any two genes can be summarized in a contingency table. For purposes of illustration, the co-occurrence of two genes, aromatase P450 and P450scc as they were sequenced in the set of 1176 cDNA libraries are presented Aromatase P450 was detected in a total of 27 libraries, and P450scc, in 13 of those 27. From the co-occurrence contingency table, the probability that the co-occurrences arose by chance using a Fisher Exact test was determined. Whereas genes known to be unrelated typically have p-values of 1.0e-2 or higher in this data set, the probability that aromatase P450 and P450scc co-occur by chance is 8.65e-12.

| Number of libraries | P450scc present | P450scc absent | Total |
|---|---|---|---|
| Aromatase present | 13 | 14 | 27 |
| Aromatase absent | 40 | 1109 | 1149 |
| Total | 53 | 1123 | 1176 |

Because multiple statistical tests were performed on each gene, the question of statistical significance and interpretation of p-values must be examined. In this case, a Bonferroni correction (dividing the desired alpha, say, P=0.01, by the number of comparisons performed) to determine a suitable p-value was applied. For n genes, n(n-1)/2 pairwise comparisons were performed; thus 40,000 genes yield 8×10e-8 pairwise comparisons and required a Bonferroni-corrected P value of 0.01/(8×10e-8) or ˜10e-11. This value is seen in the top right boxes of the following chart which shows the co-expression of the known placental steroid synthesis genes (−log P). The known genes are identified by their names (left column) and abbreviations (in the same order along the top row).

| | aromatase P-450 | P450scc | 3-beta-HSD | ADAM-12 | PLAP | PL-4 | PAPP-A | PS-beta-GE |
|---|---|---|---|---|---|---|---|---|
| aromatase P-450 | | 11 | 8.8 | 11 | 10 | 9.3 | 13 | 12 |
| P450scc | 11 | | 24 | 7.6 | 11 | 5.5 | 8.1 | 12 |
| 3-beta-HSD | 8.8 | 24 | | 4.9 | 11 | 6.6 | 0 | 12 |
| ADAM-12 | 11 | 7.6 | 4.9 | | 9.8 | 5.9 | 8 | 13 |
| PLAP | 10 | 11 | 11 | 9.8 | | 6.2 | 7.9 | 15 |
| PL-4 | 9.3 | 5.5 | 6.6 | 5.9 | 6.2 | | 9.6 | 9.7 |
| PAPP-A | 13 | 8.1 | 0 | 8 | 7.9 | 9.6 | | 9.9 |
| PS-beta-G | 12 | 12 | 12 | 13 | 15 | 9.7 | 9.9 | |

Using the LIFESEQ GOLD database (Incyte Genomics), we have identified nine genes that show strong association with known placental steroid synthesis genes. Degree of association was measured by probability values using a cutoff p-value less than 0.00001. This was followed by annotation and literature searches to insure that the genes that passed the probability test had strong association with known placental steroid synthesis genes. The process was reiterated so that an initial selection of 37,071 genes were reduced to the final nine cDNAs claimed. The following table shows the coexpression between the known placental steroid synthesis genes and the novel cDNAs (−log p). The cDNAs are identified by their Incyte numbers, and genes by their abbreviations.

| cDNA | aromatase | P450scc | 3-beta-HSD | ADAM-12 | PLAP | PL-4 | PAPP-A | PS-beta-G |
|---|---|---|---|---|---|---|---|---|
| 9049 | 6 | 6.8 | 0 | 5.5 | 10 | 4.6 | 5.5 | 9.8 |
| 196655 | 7.2 | 7.6 | 11 | 5.4 | 9.6 | 7.1 | 7.1 | 11 |
| 200386 | 14 | 7.1 | 6.6 | 10 | 9.4 | 5 | 11 | 9.2 |
| 200512 | 14 | 11 | 8.3 | 10 | 9.4 | 8.8 | 14 | 17 |
| 227944 | 10 | 8.1 | 7.3 | 8 | 7.9 | 5.4 | 6.7 | 12 |
| 251059 | 8.7 | 6.8 | 7.8 | 5.3 | 8.2 | 7.7 | 9.4 | 10 |
| 253708 | 8.7 | 3.6 | 5.9 | 8.9 | 4.1 | 10 | 7 | 8.1 |
| 401743 | 7.6 | 6 | 4.9 | 4.8 | 4.6 | 0 | 3.1 | 9 |
| 984181 | 16 | 9.9 | 0 | 9.8 | 0 | 7 | 0 | 0 |

Homology Searching of cDNA Clones and Their Deduced Proteins

The cDNAs of the Sequence Listing or the amino acid sequences encoded by the cDNA were used to query databases such as GenBank, SwissProt, BLOCKS, and the like. These databases that contain previously identified and annotated sequences or domains were searched using BLAST or BLAST 2 (Altschul et al. supra; Altschul, supra) to produce alignments and to determine which sequences were exact matches or homologs. The alignments were to sequences of prokaryotic (bacterial) or eukaryotic (animal, fungal, or plant) origin. Alternatively, algorithms such as the one described in Smith and Smith (1992, Protein Engineering 5:35-51) could have been used to deal with primary sequence patterns and secondary structure gap penalties. All of the sequences disclosed in this application have lengths of at least 49 nucleotides, and no more than 12% uncalled bases (where N is recorded rather than A, C, G, or T).

As detailed in Karlin (supra), BLAST matches between a query sequence and a database sequence were evaluated statistically and only reported when they satisfied the threshold of 10⁻²⁵ for nucleotides and 10⁻¹⁴ for peptides. Homology was also evaluated by product score calculated as follows: the % nucleotide or amino acid identity [between the query and reference sequences] in BLAST is multiplied by the % maximum possible BLAST score [based on the lengths of query and reference sequences] and then divided by 100. In comparison with hybridization procedures used in the laboratory, the electronic stringency for an exact match was set at 70, and the conservative lower limit for an exact match was set at approximately 40 (with 1-2% error due to uncalled bases).

The BLAST software suite, freely available sequence comparison algorithms (NCBI, Bethesda Md.; http://www.ncbi.nlm.nih.gov/gorf/b12.html), includes various sequence analysis programs including “blastn” that is used to align nucleic acid molecules and BLAST 2 that is used for direct pairwise comparison of either nucleic or amino acid molecules. BLAST programs are commonly used with gap and other parameters set to default settings, e.g.: Matrix: BLOSUM62; Reward for match: 1; Penalty for mismatch: −2; Open Gap: 5 and Extension Gap: 2 penalties; Gap x drop-off: 50; Expect: 10; Word Size: 11; and Filter: on. Identity or similarity is measured over the entire length of a sequence or some smaller portion thereof. Brenner et al. (1998; Proc Natl Acad Sci 95:6073-6078, incorporated herein by reference) analyzed the BLAST for its ability to identify structural homologs by sequence identity and found 30% identity is a reliable threshold for sequence alignments of at least 150 residues and 40%, for alignments of at least 70 residues.

The cDNAs of this application were compared with assembled consensus sequences or templates found in the LIFESEQ GOLD database. Component sequences from cDNA, extension, full length, and shotgun sequencing projects were subjected to PHRED analysis and assigned a quality score. All sequences with an acceptable quality score were subjected to various pre-processing and editing pathways to remove low quality 3′ ends, vector and linker sequences, polyA tails, Alu repeats, mitochondrial and ribosomal sequences, and bacterial contamination sequences. Edited sequences had to be at least 50 bp in length, and low-information sequences and repetitive elements such as dinucleotide repeats, Alu repeats, and the like, were replaced by “Ns” or masked.

Edited sequences were subjected to assembly procedures in which the sequences were assigned to gene bins. Each sequence could only belong to one bin, and sequences in each bin were assembled to produce a template. Newly sequenced components were added to existing bins using BLAST and CROSSMATCH. To be added to a bin, the component sequences had to have a BLAST quality score greater than or equal to 150 and an alignment of at least 82% local identity. The sequences in each bin were assembled using PHRAP. Bins with several overlapping component sequences were assembled using DEEP PHRAP. The orientation of each template was determined based on the number and orientation of its component sequences.

Bins were compared to one another and those having local similarity of at least 82% were combined and reassembled. Bins having templates with less than 95% local identity were split. Templates were subjected to analysis by STITCHER/EXON MAPPER algorithms that analyze the probabilities of the presence of splice variants, alternatively spliced exons, splice junctions, differential expression of alternative spliced genes across tissue types or disease states, and the like. Assembly procedures were repeated periodically, and templates were annotated using BLAST against GenBank databases such as GBpri. An exact match was defined as having from 95% local identity over 200 base pairs through 100% local identity over 100 base pairs and a homolog match as having an E-value (or probability score) of ≦1×10⁻⁸. The templates were also subjected to frameshift FASTx against GENPEPT, and homolog match was defined as having an E-value of ≦1×10⁻⁸. Template analysis and assembly was described in U.S. Ser. No. 09/276,534, filed Mar. 25, 1999.

Following assembly, templates were subjected to BLAST, motif, and other functional analyses and categorized in protein hierarchies using methods described in U.S. Ser. No. 08/812,290 and U.S. Ser. No. 08/811,758, both filed Mar. 6, 1997; in U.S. Ser. No. 08/947,845, filed Oct. 9, 1997; and in U.S. Ser. No. 09/034,807, filed Mar. 4, 1998. Then templates were analyzed by translating each template in all three forward reading frames and searching each translation against the PFAM database of hidden Markov model-based protein families and domains using the HMMER software package (Washington University School of Medicine, St. Louis Mo.; http://pfam.wustl.edu/).

The cDNA was further analyzed using MACDNASIS PRO software (Hitachi Software Engineering), and LASERGENE software (DNASTAR) and queried against public databases such as the GenBank rodent, mammalian, vertebrate, prokaryote, and eukaryote databases, SwissProt, BLOCKS, PRINTS, PFAM, and Prosite.

VII Chromosome Mapping

Radiation hybrid and genetic mapping data available from public resources such as the Stanford Human Genome Center (SHGC), Whitehead Institute for Genome Research (WIGR), and Généthon are used to determine if any of the cDNAs presented in the Sequence Listing have been mapped. Any of the fragments of the cDNA encoding the protein that have been mapped result in the assignment of all related regulatory and coding sequences mapping to the same location. The genetic map locations are described as ranges, or intervals, of human chromosomes. The map position of an interval, in cM (which is roughly equivalent to 1 megabase of human DNA), is measured relative to the terminus of the chromosomal p-arm.

VIII Hybridization Technologies and Analyses

Immobilization of cDNAs on a Substrate

The cDNAs are applied to a substrate by one of the following methods. A mixture of cDNAs is fractionated by gel electrophoresis and transferred to a nylon membrane by capillary transfer. Alternatively, the cDNAs are individually ligated to a vector and inserted into bacterial host cells to form a library. The cDNAs are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37° C. for 16 hr. The membrane is removed from the agar and consecutively placed colony side up in 10% SDS, denaturing solution (1.5 M NaCl, 0.5 M NaOH), neutralizing solution (1.5 M NaCl, 1 M Tris, pH 8.0), and twice in 2×SSC for 10 min each. The membrane is then UV irradiated in a STRATALINKER UV-crosslinker (Stratagene).

In the second method, cDNAs are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (APB). Purified nucleic acids are arranged on a nylon membrane manually or using a dot/slot blotting manifold and suction device and are immobilized by denaturation, neutralization, and UV irradiation as described above. Purified nucleic acids are robotically arranged and immobilized on polymer-coated glass slides using the procedure described in U.S. Pat. No. 5,807,522. Polymer-coated slides are prepared by cleaning glass microscope slides (Corning, Acton Mass.) by ultrasound in 0.1% SDS and acetone, etching in 4% hydrofluoric acid (VWR Scientific Products, West Chester Pa.), coating with 0.05% aminopropyl silane (Sigma-Aldrich) in 95% ethanol, and curing in a 1° C. oven. The slides are washed extensively with distilled water between and after treatments. The nucleic acids are arranged on the slide and then immobilized by exposing the array to UV irradiation using a STRATALINKER UV-crosslinker (Stratagene). Arrays are then washed at room temperature in 0.2% SDS and rinsed three times in distilled water. Non-specific binding sites are blocked by incubation of arrays in 0.2% casein in phosphate buffered saline (PBS; Tropix, Bedford Mass.) for 30 min at 60° C.; then the arrays are washed in 0.2% SDS and rinsed in distilled water as before.

Probe Preparation for Membrane Hybridization

Hybridization probes derived from the cDNAs of the Sequence Listing are employed for screening cDNAs, mRNAs, or genomic DNA in membrane-based hybridizations. Probes are prepared by diluting the cDNAs to a concentration of 40-50 ng in 45 μl TE buffer, denaturing by heating to 100° C. for five min, and briefly centrifuging. The denatured cDNA is then added to a REDIPRIME tube (APB), gently mixed until blue color is evenly distributed, and briefly centrifuged. Five μl of [³²P]dCTP is added to the tube, and the contents are incubated at 37° C. for 10 min. The labeling reaction is stopped by adding 5 μl of 0.2M EDTA, and probe is purified from unincorporated nucleotides using a PROBEQUANT G-50 microcolumn (APB). The purified probe is heated to 100° C. for five min, snap cooled for two min on ice, and used in membrane-based hybridizations as described below.

Probe Preparation for Polymer Coated Slide Hybridization

Hybridization probes derived from MRNA isolated from samples are employed for screening cDNAs of the Sequence Listing in array-based hybridizations. Probe is prepared using the GEMbright kit (Incyte Genomics) by diluting mRNA to a concentration of 200 ng in 9 μl TE buffer and adding 5 μl 5×buffer, 1 μl 0.1 M DTT, 3 μl Cy3 or Cy5 labeling mix, 1 μl RNase inhibitor, 1 μl reverse transcriptase, and 5 μl 1×yeast control mRNAs. Yeast control mRNAs are synthesized by in vitro transcription from noncoding yeast genomic DNA (W. Lei, unpublished). As quantitative controls, one set of control mRNAs at 0.002 ng, 0.02 ng, 0.2 ng, and 2 ng are diluted into reverse transcription reaction mixture at ratios of 1:100,000, 1:10,000, 1:1000, and 1:100 (w/w) to sample mRNA respectively. To examine MRNA differential expression patterns, a second set of control mRNAs are diluted into reverse transcription reaction mixture at ratios of 1:3, 3:1, 1:10, 10:1, 1:25, and 25:1 (w/w). The reaction mixture is mixed and incubated at 37° C. for two hr. The reaction mixture is then incubated for 20 min at 85° C., and probes are purified using two successive CHROMA SPIN+TE 30 columns (Clontech). Purified probe is ethanol precipitated by diluting probe to 90 μl in DEPC-treated water, adding 2 μl 1 mg/ml glycogen, 60 μl 5 M sodium acetate, and 300 μl 100% ethanol. The probe is centrifuged for 20 min at 20,800×g, and the pellet is resuspended in 12 μl resuspension buffer, heated to 65° C. for five min, and mixed thoroughly. The probe is heated and mixed as before and then stored on ice. Probe is used in high density array-based hybridizations as described below.

Membrane-Based Hybridization

Membranes are pre-hybridized in hybridization solution containing 1% Sarkosyl and 1× high phosphate buffer (0.5 M NaCl, 0.1 M NaHPO₄, 5 mM EDTA, pH 7) at 55° C. for two hr. The probe, diluted in 15 ml fresh hybridization solution, is then added to the membrane. The membrane is hybridized with the probe at 55° C. for 16 hr. Following hybridization, the membrane is washed for 15 min at 25° C. in 1 mM Tris (pH 8.0), 1% Sarkosyl, and four times for 15 min each at 25° C. in 1 mM Tris (pH 8.0). To detect hybridization complexes, XOMAT-AR film (Eastman Kodak, Rochester N.Y.) is exposed to the membrane overnight at −70° C., developed, and examined visually.

Polymer Coated Slide-Based Hybridization

Probe is heated to 65° C. for five min, centrifuged five min at 9400 rpm in a 5415° C. microcentrifuge (Eppendorf Scientific, Westbury N.Y.), and then 18 μl is aliquoted onto the array surface and covered with a coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hr at 60° C. The arrays are washed for 10 min at 45° C. in 1×SSC, 0.1% SDS, and three times for 10 min each at 45° C. in 0.1×SSC, and dried.

Hybridization reactions are performed in absolute or differential hybridization formats. In the absolute hybridization format, probe from one sample is hybridized to array elements, and signals are detected after hybridization complexes form. Signal strength correlates with probe mRNA levels in the sample. In the differential hybridization format, differential expression of a set of genes in two biological samples is analyzed. Probes from the two samples are prepared and labeled with different labeling moieties. A mixture of the two labeled probes is hybridized to the array elements, and signals are examined under conditions in which the emissions from the two different labels are individually detectable. Elements on the array that are hybridized to equal numbers of probes derived from both biological samples give a distinct combined fluorescence (Shalon WO95/35505).

Hybridization complexes are detected with a microscope equipped with an INNOVA 70 mixed gas 10 W laser (Coherent, Santa Clara Calif.) capable of generating spectral lines at 488 nm for excitation of Cy3 and at 632 nm for excitation of Cy5. The excitation laser light is focused on the array using a 20× microscope objective (Nikon, Melville N.Y.). The slide containing the array is placed on a computer-controlled X-Y stage on the microscope and raster-scanned past the objective with a resolution of 20 micrometers. In the differential hybridization format, the two fluorophores are sequentially excited by the laser. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. The sensitivity of the scans is calibrated using the signal intensity generated by the yeast control mRNAs added to the probe mix. A specific location on the array contains a complementary DNA sequence, allowing the intensity of the signal at that location to be correlated with a weight ratio of hybridizing species of 1:100,000.

The output of the photomultiplier tube is digitized using a 12-bit RTI-835H analog-to-digital (A/D) conversion board (Analog Devices, Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using the emission spectrum for each fluorophore. A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS program (Incyte Genomics).

Ix Transcript Imaging

A transcript image was performed using the LIFESEQ GOLD database (Jun01release, Incyte Genomics). The criteria for transcript imaging can be selected from category, number of cDNAs per library, library description, disease indication, clinical relevance of sample, and the like. In some transcript images, all normalized or pooled libraries, which have high copy number sequences removed prior to processing, and all mixed or pooled tissues, which are considered non-specific in that they contain more than one tissue type or more than one subject's tissue, can be excluded from the analysis. Treated and untreated cell lines and/or fetal tissue data can also be excluded where clinical relevance is emphasized. Conversely, fetal tissue may be emphasized wherever elucidation of inherited disorders or differentiation of particular adult or embryonic stem cells into tissues or organs such as nerves, heart or kidney would be aided by removing clinical samples from the analysis.

As stated in the DESCRIPTION OF THE INVENTION, transcript imaging can be used to support data from other methodologies such as GBA and microarray analysis. The number of cDNAs, the number of times the sequence was expressed, were counted and shown over the total number of cDNAs in the library. As an example, the transcript image for SEQ ID NO:9 (Incyte 984181) is shown below. The first column shows library name; the second column, the number of cDNAs sequenced in that library; the third column, the description of the library; the fourth column, absolute abundance of the transcript in the library; and the fifth column, percentage abundance of the transcript in the library.

SEQ ID NO:9
Category: Embryonic Structures

| | | | | % |
|---|---|---|---|---|
| Library | cDNAs | Description | Abundance | Abundance |
| PLACNOB01 | 3954 | placenta, | 27 | 0.6829 |
| | | neonatal, F | | |
| PLACFEP01 | 1251 | placenta, fetal, | 6 | 0.4796 |
| | | 3′ TIGR | | |
| PLACNOM01 | 1935 | placenta, fetal, M, | 2 | 0.1034 |
| | | WM | | |
| PLACFET04 | 3604 | placenta, fetal, | 3 | 0.0832 |
| | | 18 wM | | |
| PLACFER01 | 6579 | placenta, aw/ | 2 | 0.0304 |
| | | hydrocephalus, | | |
| | | fetal, 16 w | | |
| PLACFER06 | 7229 | placenta, aw/ | 2 | 0.0277 |
| | | hydrocephalus, | | |
| | | fetal, 16 w | | |
| PLACNOR01 | 4357 | placenta, aw/ | 1 | 0.0170 |
| | | hydrocephalus, | | |
| | | fetal, 16/18 wM | | |
| KIDNFET01 | 7864 | kidney, fetal, | 1 | 0.0127 |
| | | 17 wF | | |

In clinically-relevant tissue samples, SEQ ID NO:9 shows from about 2-fold (0.08) to about 22-fold (0.68) greater expression in normal placenta than in placenta associated with hydrocephalus (0.17-0.03) or in fetal kidney (0.01). Thus transcript imaging supports the use of these sequences as predicted using GBA.

X Complementary Molecules

Molecules complementary to the cDNA, from about 5 (PNA) to about 5000 bp (complement of a cDNA insert), are used to detect or inhibit gene expression. These molecules are selected using LASERGENE software (DNASTAR). Detection is described in Example VIII. To inhibit transcription by preventing promoter binding, the complementary molecule is designed to bind to the most unique 5′ sequence and includes nucleotides of the 5′ UTR upstream of the initiation codon of the open reading frame. Complementary molecules include genomic sequences (such as enhancers or introns) and are used in “triple helix” base pairing to compromise the ability of the double helix to open sufficiently for the binding of polymerases, transcription factors, or regulatory molecules. To inhibit translation, a complementary molecule is designed to prevent ribosomal binding to the MRNA encoding the protein.

Complementary molecules are placed in expression vectors and used to transform a cell line to test efficacy; into an organ, tumor, synovial cavity, or the vascular system for transient or short term therapy; or into a stem cell, zygote, or other reproducing lineage for long term or stable gene therapy. Transient expression lasts for a month or more with a non-replicating vector and for three months or more if appropriate elements for inducing vector replication are used in the transformation/expression system.

Stable transformation of appropriate dividing cells with a vector encoding the complementary molecule produces a transgenic cell line, tissue, or organism (U.S. Pat. No. 4,736,866). Those cells that assimilate and replicate sufficient quantities of the vector to allow stable integration also produce enough complementary molecules to compromise or entirely eliminate activity of the cDNA encoding the protein.

XI Protein Expression

Expression of the proteins encoded by SEQ ID NOs: 4, 6, and 7 and purification of the proteins are achieved using either a cell expression system or an insect cell expression system. The pUB6/V5-His vector system (Invitrogen, Carlsbad Calif.) is used to express protein in CHO cells. The vector contains the selectable bsd gene, multiple cloning sites, the promoter/enhancer sequence from the human ubiquitin C gene, a C-terminal V5 epitope for antibody detection with anti-V5 antibodies, and a C-terminal polyhistidine (6×His) sequence for rapid purification on PROBOND resin (Invitrogen). Transformed cells are selected on media containing blasticidin.

Alternatively, Spodoptera frugiperda (Sf9) insect cells are infected with recombinant Autographica californica nuclear polyhedrosis virus (baculovirus). The polyhedrin gene is replaced with the cDNA by homologous recombination and the polyhedrin promoter drives cDNA transcription. The protein is synthesized as a fusion protein with 6×his which enables purification as described above. Purified protein is used in an activity assay and to make antibodies.

XII Production of Antibodies

The protein expressed from SEQ ID NO:4, 6, or 7 is purified using polyacrylamide gel electrophoresis and used to immunize mice or rabbits. Antibodies are produced using standard protocols. Alternatively, the amino acid sequence of the expressed protein is analyzed using LASERGENE software (DNASTAR) to determine regions of high antigenicity. An antigenic epitope, usually found near the C-terminus or in a hydrophilic region is selected, synthesized, and used to raise antibodies. Typically, epitopes of about 15 residues in length are produced using an ABI 431A peptide synthesizer (ABI) using Fmoc-chemistry and coupled to KLH (Sigma-Aldrich) by reaction with N-maleimidobenzoyl-N-hydroxysuccinimide ester to increase antigenicity.

Rabbits are immunized with the epitope-KLH complex in complete Freund's adjuvant. Immunizations are repeated at intervals thereafter in incomplete Freund's adjuvant. After a minimum of seven weeks for mouse or twelve weeks for rabbit, antisera are drawn and tested for antipeptide activity. Testing involves binding the peptide to plastic, blocking with 1% bovine serum albumin, reacting with rabbit antisera, washing, and reacting with radio-iodinated goat anti-rabbit IgG. Methods well known in the art are used to determine antibody titer and the amount of complex formation.

XIII Purification of Naturally Occurring Protein Using Specific Antibodies

Naturally occurring or recombinant protein is purified by immunoaffinity chromatography using antibodies which specifically bind the protein encoded by SEQ ID NO:4, 6, or 7. An immunoaffinity column is constructed by covalently coupling the antibody to CNBr-activated SEPHAROSE resin (APB). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the protein is collected.

XIV Screening Molecules for Specific Binding with the cDNA or Protein

The cDNA, or fragments thereof, or the protein, or portions thereof, are labeled with ³²P-dCTP, Cy3-dCTP, or Cy5-dCTP (APB), or with BIODIPY or FITC (Molecular Probes, Eugene Oreg.), respectively. Libraries of candidate molecules or compounds previously arranged on a substrate are incubated in the presence of labeled cDNA or protein. After incubation under conditions for either a nucleic acid or amino acid sequence, the substrate is washed, and any position on the substrate retaining label, which indicates specific binding or complex formation, is assayed, and the ligand is identified. Data obtained using different concentrations of the nucleic acid or protein are used to calculate affinity between the labeled nucleic acid or protein and the bound molecule.

XV Two-Hybrid Screen

A yeast two-hybrid system, MATCHMAKER LexA Two-Hybrid system (Clontech), is used to screen for peptides that bind the protein of the invention. A cDNA encoding the protein is inserted into the multiple cloning site of a pLexA vector, ligated, and transformed into E. coli. cDNA, prepared from mRNA, is inserted into the multiple cloning site of a pB42AD vector, ligated, and transformed into E. coli to construct a cDNA library. The pLexA plasmid and pB42AD-cDNA library constructs are isolated from E. coli and used in a 2:1 ratio to co-transform competent yeast EGY48[p8op-lacZ] cells using a polyethylene glycol/lithium acetate protocol. Transformed yeast cells are plated on synthetic dropout (SD) media lacking histidine (-His), tryptophan (-Trp), and uracil (-Ura), and incubated at 30° C. until the colonies have grown up and are counted. The colonies are pooled in a minimal volume of 1×TE (pH 7.5), replated on SD/-His/-Leu/-Trp/-Ura media supplemented with 2% galactose (Gal), 1% raffinose (Raf), and 80 mg/nm 5-bromo-4-chloro-3-indolyl β-d-galactopyranoside (X-Gal), and subsequently examined for growth of blue colonies. Interaction between expressed protein and cDNA fusion proteins activates expression of a LEU2 reporter gene in EGY48 and produces colony growth on media lacking leucine (-Leu). Interaction also activates expression of β-galactosidase from the p8op-lacZ reporter construct that produces blue color in colonies grown on X-Gal.

Positive interactions between expressed protein and cDNA fusion proteins are verified by isolating individual positive colonies and growing them in SD/-Trp/-Ura liquid medium for 1 to 2 days at 30° C. A sample of the culture is plated on SD/-Trp/-Ura media and incubated at 30° C. until colonies appear. The sample is replica-plated on SD/-Trp/-Ura and SD/-His/-Trp/-Ura plates. Colonies that grow on SD containing histidine but not on media lacking histidine have lost the pLexA plasmid. Histidine-requiring colonies are grown on SD/Gal/Raf/X-Gal/-Trp/-Ura, and white colonies are isolated and propagated. The pB42AD-cDNA plasmid, which contains a cDNA encoding a protein that physically interacts with the protein, is isolated from the yeast cells and characterized.

All patents and publications mentioned in the specification are incorporated by reference herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of molecular biology or related fields are intended to be within the scope of the following claims.

TABLE 1

| SEQ | Incyte | GenBank 119 Homolog, | Embryonic | Specific |
|---|---|---|---|---|
| ID | No | Description, Length | Libs* | expression |
| 1 | 9049 | g6715702 chromosome 16 | 1 of 13 | 94.90% |
| | | clone RPCI-11_520B23 | | |
| | | (160943 nt) | | |
| 2 | 196655 | g8844111 (39945 nt) | 3 of 13 | 80.30% |
| 3 | 200386 | g5650596 (168835 nt) | 4 of 13 | 67.03% |
| 4 | 200512 | g10764174 (1131 nt) | 5 of 13 | 85.78% |
| 5 | 227944 | g7717380 chrom 21 seg | 2 of 13 | 80.30% |
| | | HS21C084 (340,000 nt) | | |
| 6 | 251059 | g4468306 clone 413H6 on | 2 of 13 | 80.90% |
| | | chromosome 6p (142599 nt) | | |
| 7 | 253708 | g5911814 clone 193G15 on | 5 of 13 | 89.50% |
| | | chromosome 4 (62493 nt) | | |
| 8 | 401743 | g3399669 chromosome 19, | 1 of 13 | 78.70% |
| | | CIT-HSP BAC (205251 nt) | | |
| 9 | 984181 | g3550114 clone 774124 on | 8 of 13 | 60.70% |
| | | chrom 1q24.1-24.3 | | |
| | | (121780 nt) | | |

*eight embryonic tissues which fell into the normalized, subtracted, or pooled categories were not counted in this analysis

What is claimed is:

1. A combination comprising a plurality of cDNAs having the nucleic acid sequences of SEQ ID NOs:1-9 and the complements of the nucleic acid sequences of SEQ ID NOs:1-9.

2. A cDNA comprising a nucleic acid sequence selected from SEQ ID NOs:4, 6, and 7 and the complements thereof.

3. A composition comprising a cDNA of claim 2.

4. A method for using a cDNA to screen a plurality of molecules to identify at least one ligand which specifically binds the cDNA, the method comprising:
a) combining a cDNA of claim 2 with a plurality of molecules under conditions to allow specific binding; and
b) detecting specific binding, thereby identifying a ligand which specifically binds the cDNA.

5. A method for using a cDNA to detect differential expression in a sample, the method comprising:
a) hybridizing a cDNA of claim 2 to nucleic acids of the sample, thereby forming one or more hybridization complexes;
b) detecting complex formation;
c) comparing complex formation with standards wherein the comparison indicates the presence of differential expression in the sample.

6. The method of claim 5 wherein the cDNA is attached to a substrate.

7. The method of claim 5 wherein the nucleic acids of the sample are amplified prior to hybridization.

8. The method of claim 5 wherein complex formation is compared with diseased and nondiseased standards and is diagnostic of a disorder associated with steroid-responsive tissues or pregnancy.

9. An expression vector comprising the cDNA of claim 2.

10. A host cell comprising the expression vector of claim 9.

11. A method for producing a protein, the method comprising:
a) culturing the host cell of claim 10 under conditions for expression of the protein; and
b) recovering the protein from cell culture.

12. A purified protein produced by the method of claim 11.

13. A composition comprising the protein of claim 12.

14. A method for using a protein to screen a plurality of molecules or compounds to identify a ligand, the method comprising:
a) combining a protein of claim 12 with the molecules or compounds under conditions to allow specific binding; and
b) detecting specific binding, thereby identifying a ligand which specifically binds the protein.

15. A method of using a protein to prepare and purify an antibody comprising:
a) immunizing a animal with a protein of claim 12 under conditions to elicit an antibody response;
b) isolating animal antibodies;
c) attaching the protein to a substrate;
d) contacting the substrate with isolated antibodies under conditions to allow specific binding to the protein;
e) dissociating the antibodies from the protein, thereby obtaining purified antibodies.

16. An isolated antibody which specifically binds a protein of claim 12.

17. A composition comprising an antibody of claim 16.

18. A method for using an antibody to detect expression in a sample, the method comprising:
a) combining the antibody of claim 16 with a sample under conditions which allow the formation of antibody:protein complexes; and
b) detecting complex formation, wherein complex formation indicates expression of the protein in the sample.

19. The method of claim 18 wherein complex formation is compared with standards and is diagnostic of a disorder associated with steroid-responsive tissues or pregnancy.

20. A method for using an antibody to immunopurify a protein comprising:
a) attaching the antibody of claim 16 to a substrate;
b) contacting the antibody with solution containing the protein, thereby forming an antibody:protein complex;
c) dissociating the antibody:protein complex; and
d) collecting the purified protein.


SEQUENCE LISTING (9 sequences)

SEQ ID NO: 1 - DNA, 586 bp, Homo sapiens
  misc_feature: Incyte ID No 009049
gggaaacagtgctaagaaggatacagtggctagaagtcgtcctgtcgtcctgcctcacagtaacatcgttaccgaattctcagcaggtgaaccaaatgaaatggtcaactgaaagccaaccaggcaaaaaatcaccatgtaccaacctatccaaacttatccatggatgaatctatccagaagacgggagttccgatgcttgtcttgctctgaatgtctgcttgtcacctgcttagggttatcgactgtgattctgggactcattgttgttctacaggacccctctgactctgtggttttctctactggattaacaatgatagccataggtgctttttttgttgtcctcactggagtgacagccctgtgtacggttacagtcgacgagaacttgcagaaaaccacgaggctaagactaggagtgatacgaaaaagcggaagtctccaaggaactacagagccttccatgactcactcaataatcgctagcacctcgctgtagttgtacattgaaccctggcatcttcgtctttggaactaagtctcctgagcattgtttttaaatagaaataaaatctggctttta

SEQ ID NO: 2 - DNA, 1122 bp, Homo sapiens
  misc_feature: Incyte ID No 196655
attttacagatggcttttggcttgcccaaggtcaccaccctctgaactgaggcgtccccaacccatgccgggagctggtggaggaagtggggaagggtttcccgcttctccactgggggtggaggtgggagaaagcattttcagggtgttttgacatttaacaaacagtatgcaaatcacatgcaaatcgcatgcaaatcaccacccatttcttaagcctcgacagcgctggcatgttctgaaataataatgttggacctgtcccttgatgatgacctgggtcgggaggtgcacggccttttccttcctccgtcttcccttcccttcccttcccttcccatctgttctcccttccttcgcccccttcctttcctctccctccctatcttccatttttctcttcttccttcacatcatttgtccaagacctccctggcctgagataaacataagttgcaaaagtggcttcctgcagcccattttccaggttgggaaactgaggcatgggcctaaggtcccacagccacaccgaggaaagcagcccccactgaatgtcaccatgcaccaaagtgcatggtcctcgtgacacaaagaggggagatgacagtggctggagttgtcagagctgctttgaggaagctccaggcctgataggcttgacagggccaaggggaactattgtggaatgtcttggccttgaatgacaggctgctgattttggatctggagaaagctcacactgactcttcctgctcccccagtctgtttttctcctgctactccatccatgtatccctgagtttgggccacagtccctccacttggtttgcatgtcccaccttctttgtttggcaagctcctattcatccttcaaagccctagcttcaatacctgctacttcatgcagccattgcttatccttcaaggagagcctacttccctcaaggaccctgctgcttccagtctcaccttctgactcactcgggatccactgaactgggaggtctgtgtctcctcccagcagagtcatcactacctttggggccgcaggatcacccagcttggaactagatacagaaatgctgttttgagagtgtactgaataaaagattacatgtttgg

SEQ ID NO: 3 - DNA, 783 bp, Homo sapiens
  misc_feature: Incyte ID No 200386
cacactcccttttctcgggtgtgtactttttgctttgtgatacatctctgcactttcagtattttccaactcatccttaaattccttctcacaacagtgtcaagagcctggacgccagccaggattgaggtcctacgggtgtttggggacctccccaagcccacgagtatcaatggcagtatcaattgtctgtgacagtgattaaggagcaaaacacttggaacccacaagactcccagaaggtgaagttaagagctcccagactcataaggttattagaacagcaaactggcaccccaaagaactttacggagacttgcaacctatcaacaagttggatgagggattaaaagccttcaacaaccaacaaccccaagcatcaaactgaaggaaacattctaaccttcacagacagactggaggctggatggggacctggctgaagacatctggagaatgaaagttaatgtaccagcttgcatttttgtgcccctagattatttttgcattttaaaataagaagcatcaaattgcgtgtctctgtgtaaaagttctagcaatttgttttaaggtgaacttattttggcttagggactacaaaaagagaaggtaattcctagggaaggaagaagagaaagaaatgaaaattagagaataagattattttgaatgacttcaggtagcgaggagtgtgtgtttgtgagtgtgtatttgagagacttggctcatgcctgtgggtcttctcttctagtatcagtgaggggagggattactgaagaagaa

SEQ ID NO: 4 - DNA, 1187 bp, Homo sapiens
  misc_feature: Incyte ID No 200512
atatacgattactatagggatttggccctcgaggcaagaaattcggcacgaggatcagaccatcagaaggatttgtataaagagtgactctcctatgaaggtaaaggccacccctcttcagttccagtgactgagatacatttttccaatcctgggggcaaatacagacacagcaagttccttcttccctttggaaatttggcagctgccttcaccagtgagcacaaagccacatttcaaaggaaactgacaaattatccccagctgccagaagaagaaatcctcactggacggcttcctgtttcctgtggttcattatctgattggctgcagggatgaaagtttttaagttcataggactgatgatcctcctcacctctgcgttttcagccggttcaggacaaagtccaatgactgtgctgtgctccatagactggttcatggtcacagtgcaccccttcatgctaaacaacgatgtgtgtgtacactttcatgaactacacttgggcctgggttgccccccaaaccatgttcagccacacgcctaccagttcacctaccgtgttactgaatgtggcatcagggccaaagctgtctctcaggacatggttatctacagcactgagatacactactcttctaagggcacgccatctaagtttgtgatcccagtgtcatgtgctgccccccaaaagtccccatggctcaccaagccctgctccatgagagtagccagcaagagcagggccacagcccagaaggatgagaaatgctacgaggtgttcagcttgtcacagtccagtcaaaggcccaactgcgattgtccaccttgtgtcttcagtgaagaagagcatacccaggtcccttgtcaccaagcaggggctcaggaggctcaacctctgcagccatctcactttcttgatatttctgaggattggtctcttcacacagatgatatgattgggtccatgtgatcctcaggtttggggtctcctgaagatgctatttctagaattagtatatagtgtacaaatgtctgacaaataagtgctcttgtgaccctcatgtgagcacttttgagaaagagaaacctatagcaacttcatgaattaagcctttttctatatttttatattcatgtgtaaacaaaaaataaaataaaattctgatcgcataaaaaaaaaaaa

SEQ ID NO: 5 - DNA, 1147 bp, Homo sapiens
  misc_feature: Incyte ID No 227944
gggacacaatcaccccaggttgagatccatggagccaggtctgtttgccaccaaggggtaaagctccattcccaccttaggagggctaggaggcagcatcgtggggccacagaaggcctgggtttgcagtcagaggacaggatgcacattccttcaagatacagacccagattgttgggcatctagttcttgggttttctgttgttgctgttccgttttgtctgtcttccctcctttgtttactagcagcctggaatttgccactttttctaaacgaagatttatggaacacttaccacacggctgacgctgcgcgaggctaaggttctaatacaccgcagctcacttaactctcgcaataccataaacgcacactgtttcatcttgaccctttcttgggaaaggtgacaggaggaggtagggaggggcaaacatcttgtgtggccccgttcccaagggtattaactggtggaataatatccgccccccaccccagtttctaatttgctgtaggctgtgacgctgtggggcaagactagggagtcctgttgaaattaggaataagtgtgctgtgagggaagggctgccttattttagagcacagattttctgaatatctattttgacaggttcgatcctctccccttcctgccttccttctgtcgattttcaatgtcttgaatggtgtccccacctgagtggcctttagagatgtgagttgtgaggcactggggaggcaggcacacgtcctccagcccaagactgccctaatttaaccagggattttctggcattcctggaacaagccctcccattttccccccaagcaggattacctcccagagggcaaaacacagccccaatagtatccacatttccctttcctgctttagcaaaaataacccactgtcctcattcatgggaaaaggccgcccaaacaaatttgttacctggaacccatttgtaacaacttcctagtttgcactgccttggagcaagcacactttgtagaggagggatttgcagttacttgggcaacaaggtaaccactgatcattacaggaagcttcagaaaccgtgggaccagtgtagaaagaatggagtatctgtccaaactaagaataaaaagaatgacacttgaaaaaaa

SEQ ID NO: 6 - DNA, 1278 bp, Homo sapiens
  misc_feature: Incyte ID No 251059
ctccttcaggccgcacacctcccaactgaagcaagagtcatacactgtcgagaacatcaagcaggatcagatgaaatctcaaaaggaaatagaaagaccgacgaggcagcaaaacaggactccctttctcctctccctgcccccatcctccttgtcaccccagcagtcccgcccagataccctcccactgaaaagtcttcactactacagcaaggagcctcccttcaaggggactggataatcaaaaatcaaaagcctgttcttccccaagagcaaagcaaggaaattctaacacctcttcaccaacccttccatatcagtgcatgcccccatacctactccttcgcccttgtttctcctccccccatctattcatctcactaaaggacataacctcaaactgtcgtatatgctctgttacttcctcccaaggggccctctgccctctcctcatccttacataccagctcagaggaacactcccaggggagcactggcaagtaaacttcacccacatgcctcccgtcaaaaaatctaaatatcttcttaccctcgtagacactttttcaggttgagtagaagcatttcctaccccttcagaaaaagctgcagaagtctctcaaattctcatatcagaaattatccctagatttggtctcccgagccccatacaatcagacaatgggcctagcttcatctcccaaattacccaacaggtctccccatcccttggcgttcagtggcgcctccatatcccatatcagccccagtcatctggagaagtcgagagggcaaacggaattctcacaactcagttaaccaaactcacacttgagatcaaaaaaccatgggacctcccttttgcccataggcactgggagagcattaggaggccagtccaaaagcaccctccttcctcagtccatttgagttaatatacggacgccctttcctcttacaaaacaggcccccttctaactctcagctagggagaatacctcccaacagtctccctcatgaggctatctcctctgccaacaagccgacccaggccctcccaaaaccccacgaaggggttctccaatcccaaataggacttgctctccaattccaaaagactctttaagcagcagtgacacttcaaaaccatcgaggtttagatctcctcactgccgagaaaggcggcctgtgtatctttttagaagaggagtgctgtttctatactaaccagtcaggactagtacaagatgctgctggacgaataaatgaaaaagcttc

SEQ ID NO: 7 - DNA, 509 bp, Homo sapiens
  misc_feature: Incyte ID No 253708
gagttgtgagggtgtgagggtcgcgttcctgctgtctggactttttctgtcccactgagacgcagctgtgtgaaatatgatttggcgaggaagatcaacatataggcctaggccgaggagaagtgtaccacctcctgagctgattgggcctatgctggagcccggtgatgaggagcctcagcaagaggaaccaccaactgaaagtcgggatcctgcacctggtcaggagagagaagaagatcagggtgcagctgagactcaagtgcctgacctggaagctgatctccaggagctgtctcagtcaaagactgggggtgaatgtggaaatggtcctgatgaccaggggaagattctgccaaaatcagaacaatttaaaatgccagaaggaggtgacaggcaaccacaggtttaaatgaagacaagctgaaacaacacaaaactgtttttatctaagatatttgacttaaaaatatcaaaataaacttttgcagctttctccaaaaaaaaaa

SEQ ID NO: 8 - DNA, 974 bp, Homo sapiens
  misc_feature: Incyte ID No 401743
aaatgtccaggcctcctcctcagatcttaaccaccatcactgaacctgaaatcctgtgtttcccaaagtgtccttgtcactgtcatgagaggatcaaggagaggaccttgttttctttccccactcacactctacaccaccacaggcccagcgagagagacacacactcaggagctctcacatgacaaatgaaggaatggaatgaagaaatgaatgatccaaaacctctttagagattggatcttggatgcagaatcctaggaggttctagccacacctgtcccttgtccttcagaggctggacatccatgtttcatccccccactacctcttgcccctaaagccaccccacctcatgcaactctgaagctctttggccaccagaggattctcagggctcctttgtcctggactgtggaactgggggcagctggtccctggggtctctgaagtcagtgtctccctcactgctcactgccatggtgtctctgcctctgcttctctgtgtccctcatcttcctcccacttcattctgactggcaagccctgtcctgcacaggttcttccccaacccctaggccttccccagaaactccctctgactaggctggctgttcaagttcccttccccactaacactgtggcctggccccacctccccaggaaataggaaaggtgcagaaatcaccctggagttgccactcctgcccaggcttcatctcgagccaatgtccccaggtcactaagagaatgagcttccactgtattcccatccagggctctttccctttgtgaggctgacctgtggacaagaccatgggacagggataggcagttcctccatccactctcataattgccaggcaagttcttctggcctctcaaatcagcctccccagtggatcaggggttagagatattcaagggtagtttcaggagcacaggactaaaatataagtactgctgattgtttg

SEQ ID NO: 9 - DNA, 3798 bp, Homo sapiens
  misc_feature: Incyte ID No 984181
gaagatacaatgcctggaaggtggaatctgggagcaaggcagctgcattcctgtggtgtgtgagccaccccctcctgtgtttgaaggcatgtatgaatgtaccaatggcttcagccctggacagccagtgtgtgctcaactgtaaccaggaacgtgaaaagcttcccatcctctgcactaaagagggcctgtggacccaggagtttaagttgtgtgagaatctgcaaggagaatgcccaccaccccctcagagctgaattctgtggagtacaaatgtgaacaaggatatgggattggtgcagtgtgttccccattgtgtgtaatcccccccagtgaccccgtgatgctacctgagaatatcactgcctgacactctggagcactggatggaacctgtcaaagtccagagcattgtgtgcactggccggcgtcaatggcacccagaccccgtcttagtccactgcatccagtcatgtgagcccttccaagcagatggttggtgtgacactatcaacaaccgagcctactgccactatgacgggggagactgctgctcttccacactctcctccaagaaggtcattccatttgctgctgactgtgacctggatgagtgcacctgccgggaccccaaggcagatagaaaatcagtaactgtgggaacaagcccctccctccactgcctcagaggcagtaagaaagagaggccgacccaggaggaaacaaagggtgaatgaagaagaacaatcatgaaatggaagaaggaggaagagcatgaaggatcttataagaaatgcaagaggatattgataggtgtgaactagttcatcaagtagcccaagtaggagagaatcataggcaaaagtttctttaaagtggcagttgattaacatggaaggggaaatatgatagatatataaggaccctcctccctcatttatattctattaaatcctatcctcaactcttgccctgctctccgctccaccccctgccaactactcagtcccacccaacttgtaaaccaataccaaaatactagaggagaagttggcagggatactgttaatacccattttgaagtggattgccatctttcagagcttgtctgctctcaactggctcttttcctttttgtgtagtttcccttaaataatgaagttagttattaattctttataagtatttaaacataattatataaatatattatatatattatattttttgctgtttactaagctaaaaattattcattgttccacacatgctgctgtgaagttcacattcaagatgaatgttgagactttgaggacagaaaggcaacttattttcccatctttctatggatgcggattggcaggttgaatgggaagtacagaaggagagagagtaattagatggaattctggatgctagcatgtaaagctaatcatctttttttttttatgacctgggagctgggcccattttatgaccaaggagatggggagttggaatggtggtactaagaggcataggaagttgagtgtgaataccatgggtgatgggtccaggagaactagactatggttcttgaatatctgtccacaaagaatatactaacttttgtcaacttctcagaactcccaactggagtcggtgagacctaggattttctgcacttccacacatgcctgttccaagtgtggctgtcagccagtcaacaagtttgtactatggcccattctctgatcaccaggattacaggaactcacacactcctcatacttggcctgtagtcctacttcttgttagaagtctccaagtctggccagtcacatgaccaagtgttgatttttctggaggaaaaattttatggaaatgatataggggaaaggtgggaggagatgaaagaacaggcaagagctgtcagggttaaatccaggcccgggcatgagaatggaagtgatcagggagactcggtccttgttccaagtctccaaagaagaccaaagtgggtcccttgagcaatgaagaatctgagataaattctcttcaagtatcatgtacaaaatctgtgagccagagattttgacttgagcaagccatggaaatgcatggagcaagggtgacactctgtggggagacagaagaatttcaactatttaatgtccattttgttgtttttaccctttcttatccaatagatggaatgcacatgaaatgaccatattaagcctctctctatttacatcccaggctcactgggatgtggtctactgcagttacattttcttgtaacggtttctggattagaccctagggaaagtgagtaaggagccagtttctgtttaacattctagttttactcattttaggaaggctgtgagtgaggcttgtctcctttaaagtttcttctccaatggaaaccaagaacagacaaaatttagagctcagctgtggtctcttctcatcttctgctcttttgctttgaccacagtttttctactcttcccatcaacactagagcaatggctgtgcaaataggaataggaaatactaccacaatgatagaaatattatccacactatcacgtagggaagaacaatatcctgaaagagaataaaacacgaataaggtgatgtacccacattaatctgtgggtttgtggaatgagggttgcaaagttattgggaaaaggaaagagcagagttcacccattcaaaaaaaaccttttgtctactaatctctagtgtaaagaaaatgtagttcagataccattcattgtcttgggtcatgcttagtgcccccaagaagacaaacatatttattcttgggattctgataggcttcaatatgcaaaggacaatggaaaagtttagacactctattttcaaaattttataaacttgttttattggggaaaatgtccaaattgctagacacattctaagttctgccttggagaatcctactttgtctgagattgaggcagaggaattgttatcctgggcattactcagctcaggaacatggagcctgtggttcatgccagtgtgtgtcttcatgcagtctctccacaagagcaacagtaagaacatttctgttttaaatttcattttaaaatattttattatctgcaattcaccactgctctgggaaagcaaaaggaaagttcctgttgtgtgtgaagagcctcttaggctataaggcttcccagccatagtcagctatagctattcagagacagcaggttcttccagtctttgttcctgggacctgatgttttgagcaactcaggtcactgataaagtggaaggactaagacactgtggtcacagatcccagcaacatcaactcacactcaatccatgtggtggtccacattctgctactcttatccacccatgtggtcattgagagcctttctcagagactcttctgtgtgtttgattgtgcccaggtggcccagggctagctggctctaacaactagcatgacagcctccaatcagaaaggcaggtaaggggacagggtgaggagaatgggcagatactgacagaaattaaagtaaagggattgtgaaagtaaagagctcttcctgattctcatcttctcttttttctattacaaggcattgaacttggcacttcctgtattctttgtgatcactattgagtgcattagttaacacccaaggggatggcttgattgggaatgtagtgaaaggagctgatctactgtattgtaatgtaaaacagctacagccagttattttgtaagattataagttgttcattaaaaaatcagcacac